(19) United States
(12) Patent Application Publication
(10) Pub. No.: US 2025/0232830 A1
(43) Pub. Date: Jul. 17, 2025

(54) METHODS AND SYSTEMS FOR VISCOSITY PREDICTION AND PROTEIN ENGINEERING

(71) Applicant: AMGEN INC., Thousand Oaks, CA (US)

(72) Inventors: Mani JAIN, Bellevue, WA (US); Lei JIA, San Diego, CA (US); Bram ESTES, Newbury Park, CA (US)

(73) Assignee: AMGEN INC., Thousand Oaks, CA (US)

(21) Appl. No.: 19/016,939

(22) Filed: Jan. 10, 2025

Related U.S. Application Data
(60) Provisional application No. 63/619,949, filed on Jan. 11, 2024.

(51) Int. Cl. G16B 15/20; G16B 30/00; G16B 40/20

(52) U.S. Cl. CPC G16B 15/20; G16B 30/00; G16B 40/20

(57) ABSTRACT

A computer-implemented method for generating one or more modified amino acid sequences comprises obtaining, via one or more processors, one or more candidate amino acid sequences and characteristic data associated with the one or more candidate amino acid sequences; classifying, via the one or more processors, the one or more candidate amino acid sequences into a first set of high-viscous amino acid sequences and a second set of low-viscous amino acid sequences using a predictive model, the classifying comprising: calculating one or more viscosity predictions based on the one or more candidate amino acid sequences and the obtained characteristic data; and determining the first set of high-viscous amino acid sequences and the second set of low-viscous amino acid sequences based on the one or more viscosity predictions and a predetermined viscosity threshold, wherein the first set of high-viscous amino acid sequences comprises at least one candidate amino acid sequence; and generating, via the one or more processors, the one or more modified amino acid sequences based on the first set of high-viscous amino acid sequences using a generative model, wherein at least one of the one or more modified amino acid sequences has a viscosity prediction lower than the predetermined viscosity threshold.

### RELATED APPLICATION

This application claims the benefit of U.S. Provisional Application No. 63/619,949 (filed on Jan. 11, 2024), which is incorporated in its entirety by reference herein.

### BACKGROUND

Viscosity is one of the primary design objectives for pharmaceutical industries because it impacts the formulation, manufacturing, stability, and delivery of protein-based therapeutics. Protein engineering can be used to develop useful proteins with lower viscosity through either synthesizing new proteins or modifying the existing protein sequence/structure. There are three major strategies for protein design: knowledge-based mutagenesis, computational protein design, and directed evolution.

### SUMMARY

In one aspect, a computer-implemented method for generating one or more modified amino acid sequences comprises obtaining, via one or more processors, one or more candidate amino acid sequences and characteristic data associated with the one or more candidate amino acid sequences; classifying, via the one or more processors, the one or more candidate amino acid sequences into a first set of high-viscous amino acid sequences and a second set of low-viscous amino acid sequences using a predictive model, the classifying comprising: calculating one or more viscosity predictions based on the one or more candidate amino acid sequences and the obtained characteristic data; and determining the first set of high-viscous amino acid sequences and the second set of low-viscous amino acid sequences based on the one or more viscosity predictions and a predetermined viscosity threshold, wherein the first set of high-viscous amino acid sequences comprises at least one candidate amino acid sequence; and generating, via the one or more processors, the one or more modified amino acid sequences based on the first set of high-viscous amino acid sequences using a generative model, wherein at least one of the one or more modified amino acid sequences has a viscosity prediction lower than the predetermined viscosity threshold.

In some embodiments, the characteristic data associated with the one or more candidate amino acid sequences comprises at least one of charge, aromatic content, or hydrophobicity score. In some embodiments, the classifying further comprises, prior to calculating the one or more viscosity predictions, generating one or more features based on the characteristic data associated with the one or more candidate amino acid sequences. In some embodiments, the classifying further comprises preprocessing the one or more features by applying one or more transformations including at least one of cleaning, centralizing, or scaling. In some embodiments, the one or more transformations include cleaning, and wherein the cleaning comprises removing zero and near-zero variance features. In some embodiments, the classifying further comprises selecting a subset of the one or more transformed features via recursive feature elimination.

In some embodiments, the predictive model comprises a random forest model. In some embodiments, the predetermined viscosity threshold is 15 centipoise. In some embodiments, each of the one or more viscosity predictions corresponds to one of the one or more candidate amino acid sequences. In some embodiments, at least one of the first set of high-viscous amino acid sequences has a viscosity prediction higher than or equal to the predetermined viscosity threshold. In some embodiments, the second set of low-viscous amino acid sequences comprises at least one candidate amino acid sequence, and wherein at least one of the second set of low-viscous amino acid sequences has a viscosity prediction lower than the predetermined viscosity threshold.

In some embodiments, the generating the one or more modified amino acid sequences using the generative model comprises: generating one or more amino acid structures based on the first set of high-viscous amino acid sequences; determining one or more surface features including one or more patches based on the one or more amino acid structures; calculating one or more patch scores associated with the one or more patches, each of the one or more patch scores corresponding to one of the one or more patches; selecting a subset of the one or more patches based on the one or more patch scores; calculating one or more contribution scores associated with one or more amino acid residues on the subset of the one or more patches, each of the one or more contribution scores corresponding to one of the one or more amino acid residues; identifying one or more non-interactive amino acid residues based on the one or more contribution scores and a predetermined contribution score threshold; and substituting the one or more non-interactive amino acid residues with one or more alternate amino acid residues to generate the one or more modified amino acid sequences. In some embodiments, the generating the one or more modified amino acid sequences using the generative model comprises: generating one or more amino acid structures based on the first set of high-viscous amino acid sequences; identifying one or more non-interactive amino acid residues based on the one or more amino acid structures; and substituting the one or more non-interactive amino acid residues with one or more alternate amino acid residues to generate the one or more modified amino acid sequences.

In some embodiments, the method for generating one or more modified amino acid sequences further comprises providing the second set of low-viscous amino acid sequences for laboratory experiments. In some embodiments, the method for generating one or more modified amino acid sequences comprising classifying the one or more modified amino acid sequences using the predictive model.

In another aspect, a computer-implemented method for determining one or more viscosity predictions associated with one or more candidate amino acid sequences comprises obtaining, via one or more processors, one or more candidate amino acid sequences; determining, via the one or more processors, characteristic data associated with the one or more candidate amino acid sequences based on the one or more candidate amino acid sequences; generating, via the one or more processors, one or more transformed features based on one or more transformations and the characteristic data associated with the one or more candidate amino acid sequences; selecting, via the one or more processors, a subset of the one or more transformed features using one or more feature elimination criteria; and determining, via the one or more processors, the one or more viscosity predictions based on the subset of the one or more transformed features, wherein each of the one or more viscosity predictions corresponds to one of the one or more candidate amino acid sequences. In some embodiments, the one or more transformations includes at least one of cleaning, centralizing, or scaling. In some embodiments, the one or more feature elimination criteria comprise recursive feature elimination.

In another aspect, a computer system for generating one or more modified amino acid sequences comprises a memory storing instructions; and one or more processors configured to execute the instructions to perform operations including: obtaining, via one or more processors, one or more candidate amino acid sequences and characteristic data associated with the one or more candidate amino acid sequences; classifying, via the one or more processors, the one or more candidate amino acid sequences into a first set of high-viscous amino acid sequences and a second set of low-viscous amino acid sequences using a predictive model, the classifying comprising: calculating one or more viscosity predictions based on the one or more candidate amino acid sequences and the obtained characteristic data; and determining the first set of high-viscous amino acid sequences and the second set of low-viscous amino acid sequences based on the one or more viscosity predictions and a predetermined viscosity threshold, wherein the first set of high-viscous amino acid sequences comprises at least one candidate amino acid sequence; and generating, via the one or more processors, the one or more modified amino acid sequences based on the first set of high-viscous amino acid sequences using a generative model, wherein at least one of the one or more modified amino acid sequences has a viscosity prediction lower than the predetermined viscosity threshold.

In yet another aspect, a non-transitory computer readable medium for use on a computer system contains computer-executable programming instructions for performing a method of generating one or more modified amino acid sequences, the method comprising: obtaining, via one or more processors, one or more candidate amino acid sequences and characteristic data associated with the one or more candidate amino acid sequences; classifying, via the one or more processors, the one or more candidate amino acid sequences into a first set of high-viscous amino acid sequences and a second set of low-viscous amino acid sequences using a predictive model, the classifying comprising: calculating one or more viscosity predictions based on the one or more candidate amino acid sequences and the obtained characteristic data; and determining the first set of high-viscous amino acid sequences and the second set of low-viscous amino acid sequences based on the one or more viscosity predictions and a predetermined viscosity threshold, wherein the first set of high-viscous amino acid sequences comprises at least one candidate amino acid sequence; and generating, via the one or more processors, the one or more modified amino acid sequences based on the first set of high-viscous amino acid sequences using a generative model, wherein at least one of the one or more modified amino acid sequences has a viscosity prediction lower than the predetermined viscosity threshold.

In another aspect, a modified amino acid sequence is generated by a computer-implemented method, wherein the computer-implemented method comprises: obtaining, via one or more processors, a candidate amino acid sequence and characteristic data associated with the candidate amino acid sequence; classifying, via the one or more processors, the candidate amino acid sequence as a high-viscous amino acid sequence using a predictive model, wherein the predictive model calculates a viscosity prediction based on the candidate amino acid sequence and the obtained characteristic data and determines the candidate amino acid sequence as the high-viscous amino acid sequence based on the viscosity prediction and a predetermined viscosity threshold; and generating, via the one or more processors, the modified amino acid sequence based on the high-viscous amino acid sequence using a generative model, wherein the generative model identifies one or more non-interactive amino acid residues and substitutes the one or more non-interactive amino acid residues with one or more alternate amino acid residues to generate the modified amino acid sequence.

### BRIEF DESCRIPTION OF DRAWINGS

The skilled artisan will understand that the figures, described herein, are included for purposes of illustration and are not limiting on the present disclosure. The drawings are not necessarily to scale, emphasis instead being placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 is a block diagram of an exemplary system 100 for generating one or more modified amino acid sequences and/or determining one or more viscosity predictions associated with one or more candidate amino acid sequences, in accordance with some embodiments of the technology described herein.

FIG. 2 is a flowchart of an exemplary method 200 for generating one or more modified amino acid sequences, in accordance with some embodiments of the technology described herein.

FIG. 3 is a diagram depicting an illustrative technique 300 for generating one or more modified amino acid sequences, according to some embodiments of the technology described herein.

FIG. 4 is a flowchart of an exemplary method 400 for determining one or more viscosity predictions associated with one or more candidate amino acid sequences, in accordance with some embodiments of the technology described herein.

FIG. 5 depicts an exemplary process 510 for training a predictive model to determine one or more viscosity predictions associated with one or more candidate amino acid sequences, and an exemplary process 520 for determining one or more viscosity predictions associated with one or more candidate amino acid sequences, in accordance with some embodiments of the technology described herein.

FIG. 6 depicts an exemplary surface patch analysis of MMAb3 Fab from PDB:7REW, in accordance with some embodiments of the technology described herein.

FIG. 7 depicts an exemplary Pymol co-crystal of MMAb3 Fab with NHP IL-13 from PDB:7REW, in accordance with some embodiments of the technology described herein.

FIG. 8 depicts parts of the exemplary Pymol co-crystal of MMAb3 Fab with NHP IL-13 from PDB:7REW, in accordance with some embodiments of the technology described herein.

FIG. 9 depicts exemplary viscosity predictions for 16 MMAb3.1 variants 910 and the comparison between the viscosity predictions and measured viscosity for 16 MMAb3.1 variants 920, in accordance with some embodiments of the technology described herein.

FIG. 10 depicts exemplary functional assessment of lead candidates with a human peripheral blood mononuclear cells (PBMC), IL-13 induced TARC assay, in accordance with some embodiments of the technology described herein.

FIG. 11 is a schematic diagram of an illustrative computing device with which aspects described herein may be implemented.

### DETAILED DESCRIPTION

While antibody-based therapeutics have a track record of successful advancement to patients, the protein sequence of the drug product may not be identical to the molecule discovered in the natural repertoire or display library. Protein engineering can often comprise refining antibody sequences from discovery repertoires into human therapeutics. For example, scientists may substitute amino acid sequences that are the sources of chemical liabilities or instability to the molecule to eliminate a problematic property while retaining both binding to target and functional activity. For many liabilities, such as oxidation, isomerization, and deamidation, collective data can provide insight into which amino acid combinations are problematic and which substitutions can mitigate the problem. Similarly, viscosity of antibodies can be reduced to allow for formulation at a relatively high concentration. Protein surface properties can be key contributors to protein instability (e.g., colloidal protein stability) such as high viscosity, for example patches of uniformly charged or hydrophobic residues on the protein surface. Conventional approaches to measure or predict viscosity of amino acid sequences involve experimentally screening candidate amino acid sequences for viscosity. However, experimental screening is resource intensive, time consuming, and expensive to perform experiments on each amino acid sequence.

Computational methods to determine viscosity of amino acid sequences (e.g., candidate amino acid sequences) are useful. However, conventional computational methods for predicting viscosity are unreliable and inaccurate. For example, some conventional computational techniques include processing an amino acid sequence using a computational model that predicts viscosity by identifying similar amino acid sequences with known viscosity. However, while two amino acid sequences may have high similarity, this does not mean that they will have similar viscosity. Accordingly, the prediction generated by such computational model may be inaccurate. Additionally, some conventional computational techniques can determine viscosity but cannot provide insight of how to modify the amino acid sequences to improve the viscosity properties of the amino acid sequences.

There is a need for accurate computational methods to predict viscosity and improve the viscosity properties of the amino acid sequences. Accordingly, the inventors have developed machine learning techniques for predicting viscosity of the amino acid sequences and generating modified amino acid sequences that improve the viscosity properties. In details, the inventors have developed machine learning techniques for predicting viscosity for different biological and/or chemical materials. Such knowledge base can be applied to engineering for protein stability (e.g., colloidal protein stability), especially for viscosity of antibodies when formulated at a relatively high concentration, e.g., ≥150 mg/mL. The methods and systems described herein can comprise a classifier that predicts from a candidate amino acid sequence whether such sequence can be high-viscous amino acid sequence. In one example, such high-viscous amino acid sequence can have a viscosity of 15 cP or more at 150 mg/mL in a standard formulation buffer at pH 5.2.

The methods and systems disclosed herein can also provide insight of how to modify the candidate amino acid sequences to improve the viscosity properties of the candidate amino acid sequences. In particular, the methods and systems disclosed herein can comprise a generative model to generate modified amino acid sequences with improved viscosity properties (e.g., lower viscosity). Additionally, the methods and systems disclosed herein can comprise classifying the one or more modified amino acid sequences using the predictive model such that modified amino acid sequences with potentially improved viscosity properties can be determined and/or classified with the predictive model. Moreover, the methods and systems disclosed herein can include a loop: from a predictive model that can determine high-viscous amino acid sequences, to a generative model that can generate modified amino acid sequences, and back to the predictive model that can classify the modified amino acid sequence. Such loop can be repeated multiple times until the outputs of the predictive model comprise no high-viscous amino acid sequence. Thus, the methods and systems disclosed herein can increase the success rate and shorten the timeline to obtain amino acid sequences with improved viscosity.

The methods and systems disclosed herein can lead to successful therapeutic engineering through designs focused on rational modulation of protein surface properties filtered through a predictive model to reduce the number of variants in the panel to a size that can be tractable for high-concentration viscosity measurement. The predictive model can be a classifier, and the key leverage gained from the combination of a predictive model and a generative model can be the ability to rationally reduce the number of candidate amino acid sequences to identify low viscosity engineered variants in a single large-scale round of production. The methods and systems disclosed herein can obtain directed measures of viscosity predictions for all candidate molecules (e.g., amino acids) and generate enough material for a more complete characterization. As a result, the methods and systems disclosed herein can both increase the chance of success and shorten the timeline to identifying the molecule with the best biophysical characteristics.

Additionally, a useful application of the methods and systems disclosed herein can include predicting viscosity of newly obtained monoclonal antibodies (mAb) sequences as soon as they are available. High-viscous mAbs can be sorted out from the low-viscous mAbs and can then be either deprioritized or proactively engineered with similar cycles of in silico testing of engineered variants with the viscosity prediction algorithm. In addition, when molecules have to be re-engineered to mitigate other molecular liabilities, the resulting sequences can be screened for low viscosity before being advanced to laboratory production. The methods and systems disclosed herein allow to screen for viscosity at the sequence level with high confidence before generating molecules, which is an empowering asset that contributes to increased efficiency and success at engineering well-behaved antibodies and shortening engineering timelines.

As used herein, the terms “amino acid” and “residue” are interchangeable and, when used in the context of a peptide or polypeptide, refer to both naturally occurring and synthetic amino acids, as well as amino acid analogs, amino acid mimetics and non-naturally occurring amino acids that are chemically similar to the naturally occurring amino acids.

A “polypeptide” is a polymer of amino acids joined together by peptide bonds. A “peptide” is a polypeptide comprising less than about 50, about 40, about 30, or about 20 amino acids.

A “naturally occurring amino acid” is an amino acid that is encoded by the genetic code, as well as those amino acids that are encoded by the genetic code that are modified after synthesis, e.g., hydroxyproline, γ-carboxyglutamate, and O-phosphoserine. An amino acid analog is a compound that has the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but will retain the same basic chemical structure as a naturally occurring amino acid.

An “amino acid mimetic” is a chemical compound that has a structure that is different from the general chemical structure of an amino acid, but that functions in a manner similar to a naturally occurring amino acid. Examples include a methacryloyl or acryloyl derivative of an amide, β-, γ-, δ-imino acids (such as piperidine-4-carboxylic acid) and the like.

A “non-naturally occurring amino acid” is a compound that has the same basic chemical structure as a naturally occurring amino acid, but is not incorporated into a growing polypeptide chain by the translation complex. “Non-naturally occurring amino acid” also includes, but is not limited to, amino acids that occur by modification (e.g., posttranslational modifications) of a naturally encoded amino acid (including but not limited to, the 20 common amino acids) but are not themselves naturally incorporated into a growing polypeptide chain by the translation complex. A non-limiting lists of examples of non-naturally occurring amino acids that can be inserted into a polypeptide sequence or substituted for a wild-type residue in polypeptide sequence include β-amino acids, homoamino acids, cyclic amino acids and amino acids with derivatized side chains. Examples include (in the L-form or D-form; abbreviated as in parentheses): citrulline (Cit), homocitrulline (hCit), Nα-methylcitrulline (NMeCit), Nα methylhomocitrulline (Nα-MeHoCit), ornithine (Orn), Nα-Methylornithine (Nα-MeOrn or NMeOrn), sarcosine (Sar), homolysine (hLys or hK), homoarginine (hArg or hR), homoglutamine (hQ), Nα methylarginine (NMeR), Nα-methylleucine (Nα-MeL or NMeL), N-methylhomolysine (NMeHoK), Nα-methylglutamine (NMeQ), norleucine (Nle), norvaline (Nva), 1,2,3,4-tetrahydroisoquinoline (Tic), Octahydroindole-2-carboxylic acid (Oic), 3-(1-naphthyl)alanine (1-Nal), 3-(2-naphthyl)alanine (2 Nal), 1,2,3,4-tetrahydroisoquinoline (Tic), 2-indanylglycine (IgI), para-iodophenylalanine (pI Phe), para-aminophenylalanine (4AmP or 4-Amino-Phe), 4-guanidino phenylalanine (Guf), glycyllysine (abbreviated “K(Nε-glycyl)” or “K(glycyl)” or “K(gly)”), nitrophenylalanine (nitrophe), aminophenylalanine (aminophe or Amino-Phe), benzylphenylalanine (benzylphe), γ carboxyglutamic acid (γ-carboxyglu), hydroxyproline (hydroxypro), p-carboxyl-phenylalanine (Cpa), α-aminoadipic acid (Aad), Nα-methyl valine (NMeVal), N-α-methyl leucine (NMeLeu), Nα methylnorleucine (NMeNle), cyclopentylglycine (Cpg), cyclohexylglycine (Chg), acetylarginine (acetylarg), α, β-diaminopropionoic acid (Dpr), α, γ-diaminobutyric acid (Dab), diaminopropionic acid (Dap), cyclohexylalanine (Cha), 4-methyl-phenylalanine (MePhe), β, β-diphenyl-alanine (BiPhA), aminobutyric acid (Abu), 4-phenyl-phenylalanine (or biphenylalanine; 4Bip), α-amino-isobutyric acid (Aib), beta-alanine, beta-aminopropionic acid, piperidinic acid, aminocaprioic acid, aminoheptanoic acid, aminopimelic acid, desmosine, diaminopimelic acid, N-ethylglycine, N ethylaspargine, hydroxylysine, allo-hydroxylysine, isodesmosine, allo-isoleucine, N methylglycine, N methylisoleucine, N-methylvaline, 4-hydroxyproline (Hyp), γ-carboxyglutamate, ε-N,N,N-trimethyllysine, ε-N-acetyllysine, O-phosphoserine, N-acetylserine, N-formylmethionine, 3 methylhistidine, 5-hydroxylysine, ω-methylarginine, 4-Amino-O-Phthalic Acid (4APA), and other similar amino acids, and derivatized forms of any of those specifically listed.

The terms “polynucleotide”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

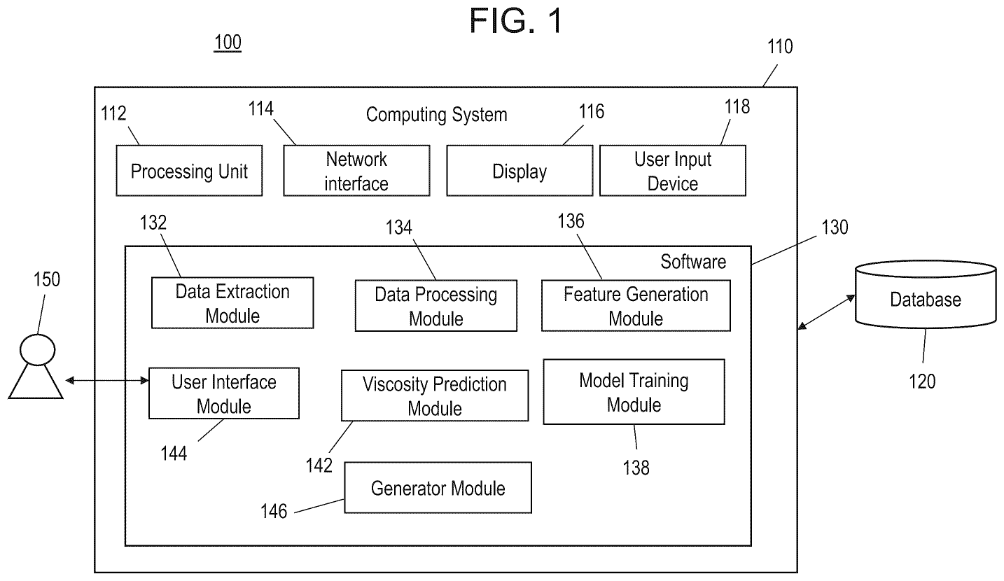

FIG. 1 is a block diagram of an exemplary system 100 for generating one or more modified protein sequences and/or determining one or more viscosity predictions associated with one or more candidate amino acid sequences, in accordance with some embodiments of the technology described herein.

System 100 includes a computing system 110 coupled to a database 120. Computing system 110 can be configured to have software 130 execute thereon to perform various functions in connection with determining one or more viscosity predictions associated with one or more candidate amino acid sequences and generating one or more modified amino acid sequences. Computing system 110 can comprise a single computing device or include multiple co-located and/or distributed computing devices communicatively coupled by one or more networks. The computing system 110 can comprise one or multiple computing devices of any suitable type. For example, the computing system 110 may be a portable computing device (e.g., laptop, a smartphone) or a fixed computing device (e.g., a desktop computer, a server). When computing system 110 includes multiple computing devices, the device(s) may be physically co-located (e.g., in a single room) or distributed across multiple physical locations. The computing system 110 may be part of a cloud computing infrastructure.

The computing system 110 may be operated by one or more user(s) 150 such as one or more researchers, health professionals, and/or other individual(s). For example, the user(s) 150 may provide one or more candidate amino acid sequences and/or characteristic data associated with the one or more candidate amino acid sequences as input to the computing system 110 (e.g., by uploading one or more files), and/or may provide user input specifying processing or other methods to be performed on one or more candidate amino acid sequences and characteristic data associated with the one or more candidate amino acid sequences.

In the example embodiment shown in FIG. 1, computing system 110 includes a processing unit 112, a network interface 114, a display 116, a user input device 118, and a software 130. Processing unit 112 includes one or more processors, each of which may be a programmable microprocessor that executes software instructions stored in memory to execute some or all of the functions of computing system 110 as described herein. Alternatively, one, some or all of the processors in processing unit 112 may be other types of processors (e.g., application-specific integrated circuits (ASICs), field-programmable gate arrays (FPGAs), etc.), and the functionality of computing system 110 as described herein may instead be implemented, in part or in whole, in hardware. Memory may include one or more physical memory devices or units containing volatile and/or non-volatile memory. Any suitable memory type or types may be used, such as read-only memory (ROM), solid-state drives (SSDs), hard disk drives (HDDs), and so on.

Network interface 114 may include any suitable hardware (e.g., front-end transmitter and receiver hardware), firmware, and/or software configured to communicate with external devices and/or systems (e.g., a client device, or one or more servers maintaining database 120) via one or more networks using one or more communication protocols. For example, network interface 114 may be or include an Ethernet interface, and/or include a wireless local area network (LAN) interface, etc.

Display 116 may use any suitable display technology (e.g., LED, OLED, LCD, etc.) to present information to a user 150, and user input device 118 may be a keyboard or other suitable input device. In some embodiments, display 116 and user input device 118 are integrated within a single device (e.g., a touchscreen display). Generally, display 116 and user input device 118 may combine to enable a user 150 to interact with user interfaces (e.g., graphical user interfaces (GUIs)) provided by computing system 110, such as those discussed in further detail below. In some embodiments, however, computing system 110 does not include display 116 and/or user input device 118, or one or both of display 116 and user input device 118 are included in another computer or system that is communicatively coupled to computing system 110 (e.g., a client device not shown in FIG. 1).

As shown in FIG. 1, software 130 includes multiple software modules for generating one or more modified protein sequences, determining one or more viscosity predictions associated with one or more candidate amino acid sequences, and/or processing one or more candidate amino acid sequences and/or characteristic data associated with the one or more candidate amino acid sequences, such as a data extraction module 132, a data processing module 134, a feature generation module 136, a model training module 138, a viscosity prediction module 142, and a generator module 146. In the embodiment of FIG. 1, the software 130 additionally includes a user interface module 144 for obtaining user input.

In some embodiments, data extraction module 132 is generally responsible for retrieving/obtaining data (e.g., one or more candidate amino acid sequences and/or characteristic data associated with the one or more candidate amino acid sequences) from the database 120. In some embodiments, data extraction module 132 retrieves training data based on user input detected by user interface module 144. In some embodiments, data extraction module 132 retrieves data based on user input detected by user interface module 144. For example, user interface module 144 may generate and/or populate a GUI, and cause display 116 to present the GUI to a user. The user may then operate user input device 118 to enter one or more candidate amino acid sequences and/or characteristic data associated with the one or more candidate amino acid sequences via the GUI, and data extraction module 132 may retrieve one or more candidate amino acid sequences and characteristic data associated with the one or more candidate amino acid sequences. In some embodiments, the database 120 includes raw data, and data extraction module 132 constructs similar data structure(s). In some embodiments, data extraction module 132 generates data in a more readily usable form.

In some embodiments, data processing module 134 is generally responsible for processing the data (e.g., one or more candidate amino acid sequences and/or characteristic data associated with the one or more candidate amino acid sequences) from the database 120 extracted via the data extraction module 132. Data processing module 134 can clean, filter, and transform the data from the database 120 using any type of computational or mathematical techniques, including machine learning techniques. Data processing module 134 can process the data based on user input detected by user interface module 144. For example, user interface module 144 may generate and/or populate a GUI, and cause display 116 to present the GUI to a user. The user may then operate user input device 118 to enter one or more instructions related to processing the data via the GUI, and data processing module 134 may process the data based on the user input. In some embodiments, the database 120 includes raw data (e.g., data without any processing steps), and data processing module 134 can process the raw data so the data can be utilized by other modules (e.g., feature generation module 130) or processes. For example, data extraction module 132 may normalize the raw data, or may generate data in a more readily usable form (e.g., a table demonstrating the characteristic data associated with the one or more candidate amino acid sequences).

In some embodiments, the feature generation module 136 obtains processed data from the database 120, the data extraction module 132, and/or the data processing module 134, and uses the processed data to generate sets of features. Such features can include any features described elsewhere herein. For example, the feature generation module 136 may generate a set of features based on one or more candidate amino acid sequences. In some embodiments, the feature generation module 136 generates a set of features by including at least some of the obtained data in the set of features. For example, the feature generation module 136 may generate the set of features to include characteristic data associated with one or more candidate amino acid sequences. For example, feature generation module 136 may generate the set of features to include a two-dimensional (2D) matrix that that stores names of each feature (e.g., charge, aromatic content, and hydrophobicity score) as y dimension, and different values as x dimension. The generated 2D matrices may be provided as input to the machine learning model. Additionally, or alternatively, the feature generation module 136 may generate a set of features including encoded data. For example, the characteristic data associated with one or more candidate amino acid sequences may be one-hot encoded. The feature generation module 136 may include additional or alternative features in the set of features, as aspects of the technology described herein are not limited in this respect.

In some embodiments, the model training module 138 is configured to train one or more models (e.g., a predictive model, a generative model) to generate one or more modified amino acid sequences. In some embodiments, the model training module 138 trains a machine learning model using one or more candidate amino acid sequences and characteristic data associated with the one or more candidate amino acid sequences. For example, the model training module 138 may obtain training data from the database 120. In some embodiments, the model training module 138 provides trained machine learning model(s) to the database 120 so the trained machine learning model(s) can be stored. Techniques for training a machine learning model are described elsewhere herein.

In some embodiments, the viscosity prediction module 142 obtains one or more sets of features from the feature generation module 136, obtains a trained machine learning model from the model training module 138 and database 120 (which may be a data store of any suitable type), and processes the obtained set(s) of features using the obtained machine learning model to obtain viscosity of one or more candidate amino acid sequences. For example, the viscosity prediction module 142 may process the set of features generated using the trained machine learning model to obtain viscosity predictions of one or more candidate amino acid sequences. The features can be generated based on characteristic data associated with the one or more candidate amino acid sequences, and such features can comprise, but are not limited to, a charge (e.g., negative charge or positive charge), an aromatic content, or a hydrophobicity score. Techniques for predicting viscosity using machine learning are described elsewhere herein. In some embodiments, the viscosity prediction is output by the viscosity prediction module 142. For example, the predicted viscosity may be output to user(s) 150 via user interface module 144. Additionally, or alternatively, the predicted viscosity may be stored in memory and/or transmitted to one or more other computing devices. In some embodiments, the viscosity prediction module 142 classifies the one or more candidate amino acid sequences into a first set of high-viscous amino acid sequences and a second set of low-viscous amino acid sequences based on the viscosity predictions.

In some embodiments, the generator module 146 obtains the first set of high-viscous amino acid sequences from the viscosity prediction module 142, obtains a trained machine learning model from the model training module 138 and database 120 (which may be a data store of any suitable type), and processes the obtained high-viscous amino acid sequences to generate one or more modified amino acid sequences. For example, after viscosity prediction module 142 processes the set of features generated using the trained machine learning model to obtain viscosity predictions of one or more candidate amino acid sequences and classify the one or more candidate amino acid sequences into a first set of high-viscous amino acid sequences and a second set of low-viscous amino acid sequences, the generator module 146 can make modifications on the first set of high-viscous amino acid sequences to generate one or more modified amino acid sequences. Techniques for generating the one or more modified amino acid sequences are described elsewhere herein. In some embodiments, the generated modified amino acid sequences are output by the generator module 146. For example, the modified amino acid sequences may be output to user(s) 150 via user interface module 144. Additionally, or alternatively, the modified amino acid sequences may be stored in memory and/or transmitted to one or more other computing devices. In some embodiments, the generated modified amino acid sequence can be input to the viscosity prediction module. In this case, the modified amino acid sequence can be classified by the viscosity prediction module as either a high-viscous amino acid sequence or a low-viscous amino acid sequence.

As shown in FIG. 1, system 100 also includes database 120. The database 120 may store model data, characteristic data associated with the one or more amino acid sequences (e.g., candidate amino acid sequences or modified amino acid sequences), and/or raw data associated with the one or more amino acid sequences. In some embodiments, software 130 obtains data from database 120 and/or user(s) 150 (e.g., by uploading data). The database 120 may be of any suitable type (e.g., database system, multi-file, flat file, etc.) and may store data in any suitable way and in any suitable format, as aspects of the technology described herein are not limited in this respect. The database 120 may be part of software 130 (not shown) or excluded from software 130, as shown in FIG. 1. The database 120 may be part of or external to computing system 110.

The stored data may have been previously uploaded by a user (e.g., user 150), and/or from one or more public data stores and/or studies. In some embodiments, a portion of the data is processed by the data processing module 134 to obtain processed data. In some embodiments, a portion of the data is processed by the feature generation module 136 to generate sets of features to be provided as input to a machine learning model. In some embodiments, a portion of the data is used to train one or more machine learning models (e.g., with the model training module 138).

User interface module 144 may be a graphical user interface (GUI), a text-based user interface, and/or any other suitable type of interface through which a user may provide input and view information generated by software 130. For example, in some embodiments, the user interface is a webpage or web application accessible through an Internet browser. In some embodiments, the user interface is a graphical user interface (GUI) of an app executing on the user's mobile device. In some embodiments, the user interface includes a number of selectable elements through which a user may interact. For example, the user interface may include dropdown lists, checkboxes, text fields, or any other suitable element.

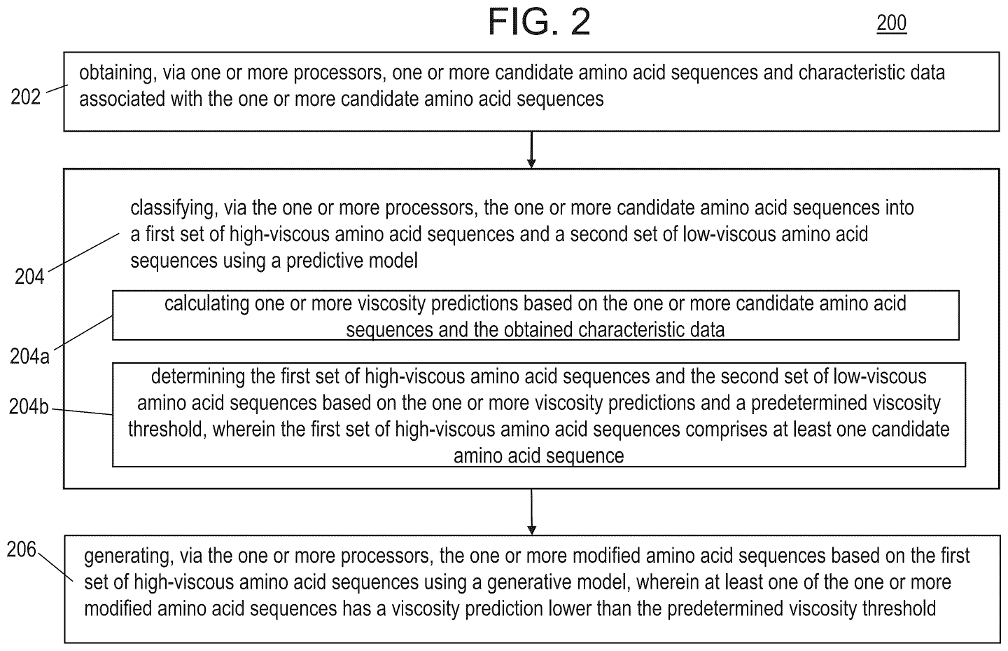

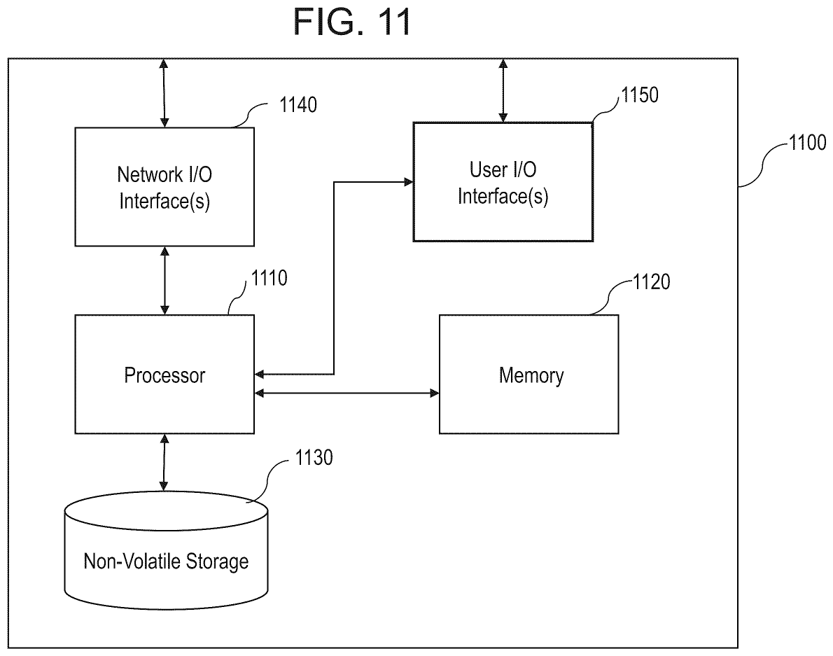

FIG. 2 is a flowchart of an illustrative method 200 for generating one or more modified protein sequences, in accordance with some embodiments of the technology described herein. One or more steps of method 200 may be performed automatically by any suitable computing system(s). For example, the step(s) may be performed by a laptop computer, a desktop computer, one or more servers, in a cloud computing environment, computer system 100, and computing device 1100 as described herein within respect to FIG. 11, and/or in any other suitable way. For example, in some embodiments, step 202 is performed automatically by any suitable computing system(s) and/or device(s). As another example, step 204 may be performed automatically by any suitable computing system(s) and/or device(s).

Step 202 may include obtaining, via one or more processors, one or more candidate amino acid sequences and characteristic data associated with the one or more candidate protein sequences. The one or more candidate amino acid sequences can be obtained from one or more nucleic acid sequences. The deoxyribonucleic acid (DNA) can encode one or more candidate amino acid sequences. For instance, the information in DNA can be transferred to a messenger ribonucleic acid (mRNA) via transcription, wherein the DNA of a gene can serve as a template for complementary base-pairing, and an enzyme (e.g., RNA polymerase) can catalyze the formation of a pre-mRNA molecule to form mature mRNA. Then the mRNA can be translated into one or more candidate amino acid sequences. In this situation, the details of the one or more candidate amino acid sequences can be determined from the sequence of DNA that encodes the one or more candidate amino acid sequences. In another example, the one or more candidate amino acid sequences can be produced by DNA cloning into plasmids and recombinant mammalian expression prior to purification. The one or more candidate amino acid sequences can comprise any type of amino acid sequences, including, but not limited to, peptides, polypeptides, fragment antibodies (e.g., Fab fragments, Fv fragments), monoclonal antibodies, antibody drug conjugates, fusion proteins, bispecific T cell engager molecules, peptibodies, and bispecific antibodies.

The characteristic data associated with the one or more candidate amino acid sequences can be determined based on the one or more candidate amino acid sequences. The characteristic data can be determined by computation via any mathematical/computational techniques based on the one or more candidate amino acid sequences. The mathematical/computational techniques can include running molecular dynamics simulations and computing biophysical features using structure (e.g., either crystal or in silico). The molecular dynamics simulations packages can comprise GROMACS (GROningen MAchine for Chemical Simulations) and LAMMPS (Large-scale Atomic/Molecular Massively Parallel Simulator). The characteristic data associated with the one or more candidate amino acid sequences can comprise at least one of a charge, an aromatic content, or a hydrophobicity score. In some embodiments, the characteristic data associated with the one or more candidate amino acid sequences can comprise an aromatic content. The characteristic data associated with the one or more candidate amino acid sequences can comprise at least one of a charge, an aromatic content, or a hydrophobicity score in one or more amino acid sequence regions including, but not limited to, fragment variable (Fv) regions of monoclonal antibodies (mAbs), complementarity-determining regions (CDRs), and frameworks. The one or more amino acid sequence regions can be selected based on the amino acid counts. For instance, in some embodiments, the one or more amino acid sequence regions comprise at least 1, 5, 10, 15, 20 or more amino acids. In some other embodiments, the one or more amino acid sequence regions comprise at most 20, 15, 10, 5 or less amino acids. The one or more amino acid sequence regions can be further filtered to select those that are likely to be informative based on their amino acid sequence patterns, for example, amino acid sequence patterns that impact the charge, aromatic content, and/or hydrophobicity score of the whole amino acid sequence.

The characteristic data can be determined by any suitable analytical/experimental techniques. Techniques for obtaining characteristic data can include, but are not limited to, mass spectrometry, chromatography, electrophoresis, spectroscopy, light obscuration, particle methods (nanoparticle/visible/micron-sized resonant mass or Brownian motion), cone and plate viscosity measurement, analytical centrifugation, imaging and imaging characterizations, and immunoassays. Example techniques for obtaining characteristic data can include reduced and non-reduced peptide mapping (which may detect chemical modifications), chromatography (such as size exclusion chromatography (SEC), ion exchange chromatography (IEX) such as cation exchange chromatography (CEX), hydrophobic interaction chromatography (HIC), affinity chromatography such as Protein A-column chromatography, or reverse phase (RP) chromatography), capillary isoelectric focusing (cIEF), capillary zone electrophoresis (CZE), free flow fractionation (FFF), or ultracentrifugation (UC), HIAC (such as for detecting subvisible particle count), MFI (such as for detecting subvisible particle count and morphology), visible inspection (visible particles), SDS-PAGE (such as for detecting fragments, covalent aggregates), color analysis (Trp Ox), rCE-SDS and nrCE-SDS (such as for detecting fragments that are partial molecules), nanoparticle sizing methods, spectroscopy methods (such as FTIR, CD, intrinsic fluorescence, or ANS dye binding), an Ellman's assay (free sulfhydryl's), SEC-MALS, HILIC (glycan map), and ELISA (such as for detecting HCP).

Step 204 may include classifying, via the one or more processors, the one or more candidate amino acid sequences into a first set of high-viscous amino acid sequences and a second set of low-viscous amino acid sequences using a predictive model. Such classification can be binary classification, where the one or more candidate amino acid sequences are divided into two categories: a first set of high-viscous amino acid sequences and a second set of low-viscous amino acid sequences. The classification can be multiclass classification, where the one or more candidate amino acid sequences are divided into three or more categories. For instance, the three categories can include a first set of high-viscous amino acid sequences, a second set of medium-viscous amino acid sequences, and a third set of low-viscous amino acid sequences.

As shown in step 204a, the classifying can comprise calculating one or more viscosity predictions based on the one or more candidate amino acid sequences and the obtained characteristic data. Prior to calculating the one or more viscosity predictions, the classifying can comprise generating one or more features based on one or more candidate amino acid sequences and the characteristic data associated with the one or more candidate amino acid sequences. The characteristic data associated with the one or more candidate amino acid sequences can comprise at least one of amino acid counts, charge, aromatic content, hydrophobicity score, or status of glycosylation and covalent disulfide bond formation. The one or more candidate amino acid sequences and/or the characteristic data associated with the one or more candidate amino acid sequences can be input into the predictive model, and then one or more features can be generated based on the one or more candidate amino acid sequences and/or the characteristic data associated with the one or more candidate amino acid sequences. In this case, the one or more features can be used to calculating one or more viscosity predictions. Each of the one or more viscosity predictions corresponds to one of the one or more candidate amino acid sequences.

As shown in step 204b, the classifying can comprise determining the first set of high-viscous amino acid sequences and the second set of low-viscous amino acid sequences based on the one or more viscosity predictions and a predetermined viscosity threshold. The first set of high-viscous amino acid sequences can comprise at least one candidate amino acid sequence. In some embodiments, the first set of high-viscous amino acid sequences comprises at least 1, 5, 10, 15, 20, 30, 40, 50, 60, 70, 80, 90, 100 or more candidate amino acid sequences. In some embodiments, the first set of high-viscous amino acid sequences comprises at most 100, 90, 80, 70, 60, 50, 40, 30, 20, 10, 5, or less candidate amino acid sequences. In some embodiments, the second set of low-viscous amino acid sequences comprises at least 1, 5, 10, 15, 20, 30, 40, 50, 60, 70, 80, 90, 100 or more candidate amino acid sequences. In some embodiments, the second set of low-viscous amino acid sequences comprises at most 100, 90, 80, 70, 60, 50, 40, 30, 20, 10, 5, or less candidate amino acid sequences. If viscosity predictions of a subset of the candidate amino acid sequences are higher than or equal to a predetermined viscosity threshold, then the subset of candidate amino sequences can be classified as the first set of high-viscous amino acid sequences. If viscosity predictions of a subset of the candidate amino acid sequences are lower than a predetermined viscosity threshold, then the subset of candidate amino sequences can be classified as the second set of low-viscous amino acid sequences. The predetermined viscosity threshold can be 15 centipoises. The predetermined viscosity threshold can be at least 1, 2, 3, 4, 5, 6, 7, 8, 9, 10, 11, 12, 13, 14, 15, 16, 17, 18, 19, 20, or more centipoises. The predetermined viscosity threshold can be at most 20, 19, 18, 17, 16, 15, 14, 13, 12, 11, 10, 9, 8, 7, 6, 5, 4, 3, 2, 1, or less centipoise. In one example, viscosity predictions of the high-viscous amino acid sequences are higher than or equal to 1, 2, 3, 4, 5, 6, 7, 8, 9, 10, 11, 12, 13, 14, 15, 16, 17, 18, 19, or 20 centipoises. In another example, viscosity predictions of the low-viscous amino acid sequences are lower than 1, 2, 3, 4, 5, 6, 7, 8, 9, 10, 11, 12, 13, 14, 15, 16, 17, 18, 19, or 20 centipoises.

At least one of the first set of high-viscous amino acid sequences can have a viscosity prediction higher than or equal to the predetermined viscosity threshold. In some embodiments, all of the first set of high-viscous amino acid sequences have a viscosity prediction higher than or equal to the predetermined viscosity threshold. In some embodiments, at least 10%, 20%, 30%, 40%, 50%, 60%, 70%, 80%, 90% or more of the first set of high-viscous amino acid sequences have a viscosity (e.g., viscosity measured by wet lab procedures) higher than or equal to the predetermined viscosity threshold. In some embodiments, at most 90%, 80%, 70%, 60%, 50%, 40%, 30%, 20%, 10% or less of the first set of high-viscous amino acid sequences have a viscosity higher than or equal to the predetermined viscosity threshold. Such viscosity can be measured via wet lab procedures (e.g., laboratory experiments). Such wet lab procedures can comprise cone and plate viscosity measurements. For instance, in one example, cone and plate viscosity measurements were performed at 25° C. on an Anton Paar Rheometer.

In some embodiments, the classifying comprises obtaining, using the predictive model, a first set of high-viscous amino acid sequences, a second set of medium-viscous amino acid sequences, and a third set of low-viscous amino acid sequences based on the one or more viscosity predictions and two predetermined viscosity thresholds, wherein a first predetermined viscosity threshold is higher than a second predetermined viscosity threshold. The first set of high-viscous amino acid sequences can comprise at least one candidate amino acid sequence. In some embodiments, the first set of high-viscous amino acid sequences comprises at least 1, 5, 10, 15, 20, 30, 40, 50, 60, 70, 80, 90, 100 or more candidate amino acid sequences. In some embodiments, the first set of high-viscous amino acid sequences comprises at most 100, 90, 80, 70, 60, 50, 40, 30, 20, 10, 5, or less candidate amino acid sequences.

In this situation, if viscosity predictions of a subset of the candidate amino acid sequences are higher than or equal to the first predetermined viscosity threshold, then the subset of candidate amino sequences can be classified as the first set of high-viscous amino acid sequences. If viscosity predictions of a subset of the candidate amino acid sequences are lower than the first predetermined viscosity threshold but higher than or equal to the second predetermined viscosity threshold, then the subset of candidate amino sequences can be classified as the second set of medium-viscous amino acid sequences. If viscosity predictions of a subset of the candidate amino acid sequences are lower than the second predetermined viscosity threshold, then the subset of candidate amino sequences can be classified as the third set of low-viscous amino acid sequences. At least one of the first set of high-viscous amino acid sequences can have a viscosity higher than or equal to the first predetermined viscosity threshold. In some embodiments, all of the first set of high-viscous amino acid sequences have a viscosity higher than or equal to the predetermined viscosity threshold. In some embodiments, at least 10%, 20%, 30%, 40%, 50%, 60%, 70%, 80%, 90% or more of the first set of high-viscous amino acid sequences have a viscosity higher than or equal to the first predetermined viscosity threshold. In some embodiments, at most 90%, 80%, 70%, 60%, 50%, 40%, 30%, 20%, 10% or less of the first set of high-viscous amino acid sequences have a viscosity higher than or equal to the first predetermined viscosity threshold. In case the one or more candidate amino acid sequences are classified as four or more categories, the classifying can be based on the one or more viscosity predictions and three or more predetermined viscosity thresholds.

The classifying can further comprise, prior to calculating the one or more viscosity predictions generating one or more features based on the characteristic data associated with the one or more candidate amino acid sequences. The number of the one or more features can be at least 1, 5, 10, 20, 30, 40, 50, 60, 70, 80, 90, 100, 110, 130, 150, 160, 180, 200, 210, 230, 250, 260, 280, 300, or more. The number of the one or more features can be at most 300, 280, 260, 250, 230, 210, 200, 180, 160, 150, 130, 110, 100, 90, 80, 70, 60, 50, 40, 30, 20, 10, or less.

The method may further comprise preprocessing the one or more features by applying one or more transformations including at least one of cleaning, centralizing, or scaling. The one or more transformations can include cleaning. The cleaning can comprise removing zero and near-zero variance features. The cleaning can comprise removing duplicated features and filtering the one or more features based on one or more filtering criteria. The one or more filtering criteria can comprise any statistical or mathematical criteria, including, but not limited to, variance threshold (e.g., features with very low variance (constant or near-constant values) can be removed), correlation threshold (e.g., some of the features that are highly correlated, which are redundant, can be removed), and missing value threshold (e.g., features with a high percentage of missing values can be removed). The transformations can comprise binarization. For instance, feature values greater than 0 can be set to 1, such that feature values are either 0 or 1. In other embodiments, a smoothing function may be implemented (e.g., to provide more granular values) instead of binarization to 0 or 1.

The classifying can further comprise selecting a subset of the one or more transformed features via recursive feature elimination. The recursive feature elimination can comprise a feature selection method that fits a model and removes the weakest feature (or features) until the specified number of features is reached. The selecting a subset of the one or more transformed features can comprise any feature selection technique/algorithm. The feature selection technique/algorithm can comprise univariate selection, recursive feature elimination, principal component analysis, and feature importance. The subset of the one or more transformed features can be used with the predictive model to calculate one or more viscosity predictions and determine the first set of high-viscous amino acid sequences and the second set of low-viscous amino acid sequences.

The predictive model can comprise a random forest model. A random forest model can comprise a machine learning method that combines multiple decision trees to improve accuracy and reduce overfitting. The random forest model can build numerous decision trees on random subsets of the data and averaging their predictions (for regression) or taking a majority vote (for classification), making it robust and less sensitive to individual tree errors. The predictive model can comprise any type of mathematical, statistical, or machine learning model, including, but not limited to, a supervised machine learning model, an unsupervised machine learning model, a reinforcement learning model, a regression model (e.g., a logistic regression model, a multinomial logistic regression model), a support vector machine model, a multilayer perceptron model, a random forest model, a natural language processing model, a neural network model, a cluster model, and a dimensionality reduction model.

The machine learning model(s), such as a predictive model or a generative model, may be created and trained based upon example data (e.g., one or more training amino acid sequences) inputs or data in order to make valid and reliable predictions or classifications for new inputs, such as testing level or production level data (e.g., one or more candidate amino acid sequences) or inputs. In supervised machine learning, a machine learning program operating on a server, computing device, or otherwise processor(s), may be provided with example inputs (or features) and their associated, or observed, outputs (e.g., labels) in order for the machine learning program or algorithm to determine or discover rules, relationships, patterns, or otherwise machine learning “models” that map such inputs (or features) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output. In unsupervised machine learning, the server, computing device, or otherwise processor(s), may be used to find its own structure in unlabeled example inputs, where, for example multiple training iterations are executed by the server, computing device, or otherwise processor(s) to train multiple generations of models until a satisfactory model, e.g., a model that provides sufficient performance when given test level or production level data or inputs, is generated. Supervised learning and/or unsupervised machine learning may also comprise retraining, relearning, or otherwise updating models with new, or different, information, which may include information received, ingested, generated, or otherwise used over time. The disclosures herein may use one or both of such supervised or unsupervised machine learning techniques.

The predictive model can be trained based on one or more training amino acid sequences. The method of training the predictive model can comprise obtaining, via one or more processors, one or more training amino acid sequences and one or more training viscosities associated with the one or more training amino acid sequences; determining, via the one or more processors, characteristic data associated with the one or more training amino acid sequences based on the one or more training amino acid sequences; training the predictive model, via the one or more processors, based on the characteristic data associated with the one or more training amino acid sequences and the one or more training viscosities. Such training viscosities can be obtained via wet lab procedures (e.g., laboratory experiments) and can be considered as ground truths related to the one or more training amino acid sequences. Wet lab procedures of measuring viscosity can further comprise normalizing each of the samples to a uniform concentration in matching formulations. The viscosity of the samples can then be measured using a cone and plate dynamic viscometer device. The measurement of the torque can rotate the cone with samples of varying viscosities occupying the space between the cone and the plate.

In some embodiments, the machine learning model (e.g., a predictive model or generative model) can be trained using a supervised or unsupervised machine learning program or algorithm. The machine learning program or algorithm may employ a neural network, which may be a deep learning neural network. The machine learning programs or algorithms may also include regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques. In some embodiments, the artificial intelligence and/or machine learning based algorithms may be included as a library or package executed on imaging server. For example, libraries may include the TENSORFLOW based library, the PYTORCH library, and/or the SCIKIT-LEARN Python library. Additionally, or alternatively, a machine learning algorithm used to train the machine learning classifier may include by way of non-limiting example, any of K-means, BIRCH, Gaussian Mixture, and/or DBSCAN and OPTICS algorithm(s). In some embodiments, training the machine learning model can comprise adjusting weights of the model of the machine learning model to learn patterns in existing data (such as identifying and/or selecting features) in order to facilitate making predictions, classifications, or identification for subsequent data.

The one or more training amino acid sequences can be obtained from one or more nucleic acid sequences. The deoxyribonucleic acid (DNA) can encode one or more training amino acid sequences. For instance, the information in DNA can be transferred to a messenger ribonucleic acid (mRNA) via transcription, wherein the DNA of a gene can serve as a template for complementary base-pairing, and an enzyme (e.g., RNA polymerase) can catalyze the formation of a pre-mRNA molecule to form mature mRNA. Then the mRNA can be translated into one or more training amino acid sequences. In this situation, the details of the one or more training amino acid sequences can be determined from the sequence of DNA that encodes the one or more candidate amino acid sequences. In another example, the one or more training amino acid sequences can be produced by DNA cloning into plasmids and recombinant mammalian expression prior to purification. The one or more training viscosity predictions can be ground truth obtained via wet lab procedures.

The one or more training amino acid sequences can be used to determine characteristic data associated with the one or more training amino acid sequences. The characteristic data associated with the one or more training amino acid sequences can comprise at least one of a charge, an aromatic content, or a hydrophobicity score. The characteristic data associated with the one or more training amino acid sequences can comprise an aromatic content. The characteristic data associated with the one or more training amino acid sequences can comprise at least one of a charge, an aromatic content, or a hydrophobicity score in one or more amino acid sequence regions (e.g., Fv regions of mAbs, CDRs, frameworks). The one or more amino acid sequence regions can be selected based on the amino acid counts. For instance, in some embodiments, the one or more amino acid sequence regions comprise at least 1, 5, 10, 15, 20 or more amino acids. In some other embodiments, the one or more amino acid sequence regions comprise at most 20, 15, 10, 5 or less amino acids. The one or more amino acid sequence regions can be further filtered to select those that are likely to be informative based on their amino acid sequence patterns, for example, amino acid sequence patterns that impact the charge, aromatic content, and/or hydrophobicity score of the whole amino acid sequence. The characteristic data can be determined by computation via any mathematical/computational techniques based on the one or more training amino acid sequences. The characteristic data can be determined by any suitable analytical/experimental techniques as described elsewhere herein.

The method of training the predictive model can further comprise a model selection step and a parameter tuning step. The method of training the predictive model can train one or more predictive models, and the one or more predictive models can comprise any type of mathematical, statistical, or machine learning model, as described herein. In this situation, the model selection step can comprise selecting a subset of predictive models out of one or more predictive models based on one or more selection criteria. The one or more selection criteria can comprise confusion matrix, precision, recall, and receiver operating characteristic (ROC) curve. The confusion matrix can demonstrate the number of times instances of one class are classified as another class. The precision can demonstrate the accuracy of positive predictions. The recall can demonstrate the ratio of positive instances that are correctly detected by the classification process. The ROC curve can plot the true positive rate. Cross-validation can be used for the parameter tuning step. For instance, training data (e.g., one or more training amino sequences) can be split into K subsets (folds) for K-fold cross-validation. Data from K-1 of the folds may be used as training data for the predictive models, and the held-out fold may be used as testing data. K can be 1, 2, 3, 4, 5, 6, 7, 8, 9, or 10.

Step 206 can comprise generating, via the one or more processors, the one or more modified amino acid sequences based on the first set of high-viscous amino acid sequences using a generative model, wherein at least one of the one or more modified amino acid sequences has a viscosity prediction lower than the predetermined viscosity threshold. In some embodiments, at least 10%, 20%, 30%, 40%, 50%, 60%, 70%, 80%, 90% or more of the one or more modified amino acid sequences have viscosity predictions lower than the predetermined viscosity threshold. In some embodiments, at most 90%, 80%, 70%, 60%, 50%, 40%, 30%, 20%, 10% or less of the one or more modified amino acid sequences have viscosity predictions lower than the predetermined viscosity threshold.

The generating one or more modified amino acid sequences using the generative model can comprise generating one or more amino acid structures based on the first set of high-viscous amino acid sequences. Each of the one or more amino acid structures can correspond to one high-viscous amino acid sequence in the first set of high-viscous amino acid sequences. The amino acid structure can comprise a three-dimensional (3D) structure corresponding to a linear chain of amino acid sequence. The 3D structure can have a single polypeptide chain backbone with one or more amino acid sequence secondary structures or amino acid sequence domains. The generating the one or more modified amino acid sequences using the generative model can comprise generating one or more amino acid structures based on the first set of high-viscous amino acid sequences; identifying one or more non-interactive amino acid residues based on the one or more amino acid structures; and substituting the one or more non-interactive amino acid residues with one or more alternate amino acid residues to generate the one or more modified amino acid sequences.

The generating the one or more modified amino acid sequences using the generative model can comprise generating one or more amino acid structures based on the first set of high-viscous amino acid sequences; determining one or more surface features including one or more patches based on the one or more amino acid structures; calculating one or more patch scores associated with the one or more patches, each of the one or more patch scores corresponding to one of the one or more patches; selecting a subset of the one or more patches based on the one or more patch scores; calculating one or more contribution scores associated with one or more amino acid residues on the subset of the one or more patches, each of the one or more contribution scores corresponding to one of the one or more amino acid residues; identifying one or more non-interactive amino acid residues based on the one or more contribution scores and a predetermined contribution score threshold; and substituting the one or more non-interactive amino acid residues with one or more alternate amino acid residues to generate the one or more modified amino acid sequences.

The generating can comprise determining one or more surface features including one or more patches based on the one or more amino acid structures. The surface features can further comprise any features related to amino acid surface structures, including, but not limited to, a position of an amino acid residue in a paratope, a shape of an amino acid residue or a paratope (e.g., the shape may play a role in matching the contours of the paratope with the epitope), and charge (e.g., positive or negative charge). The one or more patches can comprise one or more areas on the surface of the amino acid structures. The one or more areas can be connected with each other via outer edges. The one or more areas may not be connected with each other (e.g., one or more areas are isolated). The generating can comprise generating amino acid sequence aggregation propensity surfaces and performing residue-based property predictions including binding energy, thermal stability, solvent-accessible surface area, hydrophilicity, and hydrophobicity. In some embodiments, cysteine scanning automatically identifies potential mutations that can result in disulfide bridges. In this situation, identifying positions in the amino acid sequences where the introduction of cysteine residues encourages the formation of stabilizing disulfide bonds. The cysteine scanning can combine a physics based implicit solvent scoring function with a knowledge-based scoring function derived from an analysis of the geometries of disulfide bonds in amino acid structures available in the protein data bank (PDB). Relative weights can be assigned to the terms that comprise scoring function using an algorithm and find that the native disulfide in the wild-type proteins is scored on average (e.g., within the top 6% of the reasonable pairs of residues that can form a disulfide bond). In some other embodiments, reactive hot spots prone to proteolysis, glycosylation, deamidation, and oxidation are detected.

The generating can comprise calculating one or more patch scores associated with the one or more patches, each of the one or more patch scores corresponding to one of the one or more patches. Each of the one or more patch scores can represent predicted impact of such patch on the characteristics data of the amino acid sequence. The patches with larger size and “patch scores” can be visually inspected on the surface of the structure for compactness of residues with significant impact. Visual inspection can comprise analyzing the size and shape of the patch for concentrated versus dispersed charge intensity. In some embodiments, visual inspection can comprise analyzing the size and shape of the patch for concentrated versus dispersed charge intensity by research personnel or scientists. The generating can comprise selecting a subset of the one or more patches based on the one or more patch scores. The patch score can be determined based on the patch size. In one example, the patch size is 500 square angstroms, and the patch score is 500. The patch score can be at least 10, 50, 100, 200, 300, 400, 500, 600, 700, 800, 900, 1000, or more. The patch score can be at most 1000, 900, 800, 700, 600, 500, 400, 300, 200, 100, 50, 10, or less. The number of the subset of the one or more patches can be at least 1, 5, 10, 20, 30, 40, 50, 60, 70, 80, 90, 100, or more. The number of the subset of the one or more patches can be at most 100, 90, 80, 70, 60, 50, 40, 30, 20, 10, or less.

The generating can comprise calculating one or more contribution scores associated with one or more amino acid residues on the subset of the one or more patches, each of the one or more contribution scores corresponding to one of the one or more amino acid residues. The contribution score can demonstrate protein-protein binding affinity. The contribution score can be obtained based on binding free energy.

The generating can comprise identifying one or more non-interactive amino acid residues based on the one or more contribution scores and a predetermined contribution score threshold. The contribution score threshold can be at least 1, 10, 50, 100, 500, 1000 or more kcal/mol. The contribution score threshold can be at most 1000, 500, 100, 50, 1, or less kcal/mol. The non-interactive amino acid residues can comprise the amino acid residue that has the contribution scores lower than a predetermined contribution score. In some embodiments, the non-interactive amino acid residue represents the residue that does not contribute to heavy chain structure or lower chain structure. The non-interactive amino acid residues can be virtually confirmed. Interactive amino acid residues versus non-interactive amino acid residues can be determined by proximity and secondarily by the rotamer orientation of the residue and the compatibility of the proximate elements of the residues. For instance, where a strong interaction is probable, residue pairs of complementary hydrophobicity or positive and negative charge can be close together and oriented towards each other.

The generating can comprise substituting the one or more non-interactive amino acid residues with one or more alternate amino acid residues to generate the one or more modified amino acid sequences. The alternate amino acid residues can comprise the amino acid residue that has the contribution scores higher than or equal to a predetermined contribution score. In some embodiments, the amino acid residue represents the residue that contributes to heavy chain structure or lower chain structure. In some embodiments, the alternate amino acid residues do not have immunogenicity risk. For instance, modified amino acid sequences can be run through immunogenicity prediction algorithms, assessed for introducing T-cell epitopes, or generated and tested in the lab for pre-ADA or sequence based immunogenicity. Wet lab procedures including high throughput and low throughput can be implemented to determine immunogenicity risk.

The computer-implemented method can further comprise providing the second set of low-viscous amino acid sequences for laboratory experiments. The laboratory experiments can include any wet lab analytical techniques to determine the viscosity of the second set of low-viscous amino acid sequences. The laboratory experiments can comprise any wet lab procedures to produce and/or manufacture one or more amino acid sequences (e.g., the second set of low-viscous amino acid sequences). The laboratory experiments can comprise additional liability assessment of the second set of low-viscous amino acid sequences. Such laboratory experiments can comprise, but are not limited to, mass spectrometry, chromatography, electrophoresis, spectroscopy, light obscuration, particle methods (nanoparticle/visible/micron-sized resonant mass or Brownian motion), cone and plate viscosity measurement, analytical centrifugation, imaging and imaging characterizations, and immunoassays. Example wet lab techniques can include reduced and non-reduced peptide mapping (which may detect chemical modifications), chromatography (such as size exclusion chromatography (SEC), ion exchange chromatography (IEX) such as cation exchange chromatography (CEX), hydrophobic interaction chromatography (HIC), affinity chromatography such as Protein A-column chromatography, or reverse phase (RP) chromatography), capillary isoelectric focusing (cIEF), capillary zone electrophoresis (CZE), free flow fractionation (FFF), or ultracentrifugation (UC), HIAC (such as for detecting subvisible particle count), MFI (such as for detecting subvisible particle count and morphology), visible inspection (visible particles), SDS-PAGE (such as for detecting fragments, covalent aggregates), color analysis (Trp Ox), rCE-SDS and nrCE-SDS (such as for detecting fragments that are partial molecules), nanoparticle sizing methods, spectroscopy methods (such as FTIR, CD, intrinsic fluorescence, or ANS dye binding), an Ellman's assay (free sulfhydryl's), SEC-MALS, HILIC (glycan map), and ELISA (such as for detecting HCP). At least one of the second set of low-viscous amino acid sequences can have a viscosity lower than the predetermined viscosity threshold.

The computer-implemented method can further comprise classifying the one or more modified amino acid sequences via the predictive model. Such classification process can be similar to the classification process for the one or more candidate amino acid sequences, as described elsewhere herein. For instance, the classifying can comprise classifying the one or more modified amino acid sequences into a first set of high-viscous amino acid sequences and a second set of low-viscous amino acid sequences. In some embodiments, the first set of high-viscous amino acid sequences does not contain any modified amino acid sequences. In some cases that viscosity predictions of the modified amino acid sequences are higher than the predetermined viscosity threshold, the method can further comprise generating, via the one or more processors, the one or more further-modified amino acid sequences based on the modified amino acid sequences using a generative model, wherein at least one of the one or more further-modified amino acid sequences has a viscosity prediction lower than the predetermined viscosity threshold. In some embodiments, the one or more further-modified amino acid sequences can be provided to the predictive model and classified into a first set of high-viscous amino acid sequences and a second set of low-viscous amino acid sequences via the predictive model. The computer-implemented method can comprise a loop: from a predictive model that can determine high-viscous amino acid sequences, to a generative model that can generate modified amino acid sequences, and back to the predictive model that can classify the modified amino acid sequence. Such loop can be repeated multiple times until the outputs of the predictive model comprise no high-viscous amino acid sequence.

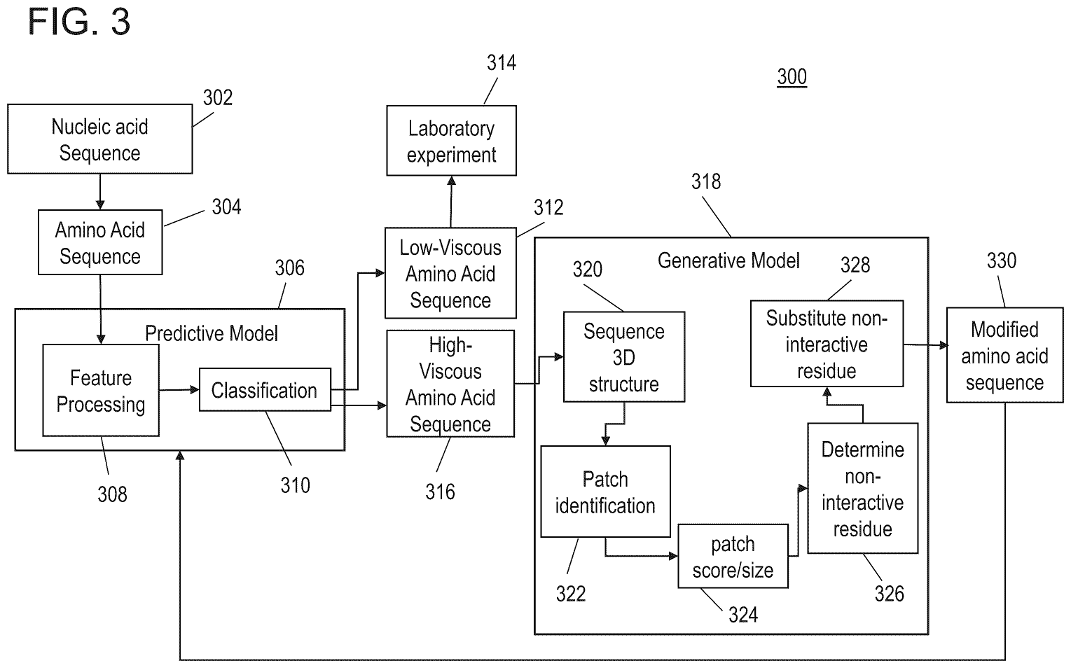

FIG. 3 is a diagram depicting an illustrative technique 300 for generating one or more modified amino acid sequences, according to some embodiments of the technology described herein.

The nucleic acid sequence 302 can comprise deoxyribonucleic acid (DNA) sequence, ribonucleic acid (RNA) sequence or any hybrid or fragment thereof. A messenger RNA (mRNA) obtained from a chromosomal DNA sequence can specify the amino acid sequence 304. One or more features can be generated via feature processing 308 based on the characteristic data (not shown in FIG. 3) associated with the amino acid sequence 304. Feature processing 308 can preprocess the one or more features by applying one or more transformations including at least one of cleaning, centralizing, or scaling. Feature processing 308 can select a subset of one or more features via mathematical and/or statistical techniques (e.g., recursive feature elimination). The feature processing 308 and/or the classification 310 can be part of the predictive model 306. The feature processing 308 may not be part of the predictive model 306 but the classification 310 can be part of the predictive model 306. The feature processing 308 can be part of the predictive model 306 but the classification 310 may not be part of the predictive model 306. The classification 310 can classify, via the one or more processors, the amino acid sequence 304 as either a high-viscous amino acid sequence or a low-viscous amino acid sequence. Such classification 310 can comprise calculating a viscosity prediction based on the amino acid sequence 304, one or more features, and/or the obtained characteristic data; and obtaining as either a high-viscous amino acid sequence or a low-viscous candidate amino acid sequence based on the viscosity prediction and a predetermined viscosity threshold.

In case the amino acid sequence 304 is classified via the predictive model 306 as a low-viscous candidate amino acid sequence 312, the amino acid sequence can be provided to further laboratory experiment 314. In case the amino acid sequence 304 is classified via the predictive model 306 as a high-viscous amino acid sequence 316, the amino acid sequence 304 can be provided to a generative model 318 to generate modified amino acid sequence 330. Sequence 3D structure 320 can be generated based on the high-viscous amino acid sequence 316. Patch identification 322 can identify one or more patches associated with the amino acid sequence 304 based on the sequence 3D structure 320. Patch scores/sizes 324 can be calculated for each of the one or more patches, and a subset of the one or more patches can be selected based on the one or more patch scores/sizes 324. One or more contribution scores associated with one or more amino acid residues on the subset of the one or more patches can be calculated. One or more non-interactive amino acid residues can be determined 326 based on the one or more contribution scores and a predetermined contribution score threshold. The one or more non-interactive amino acid residues can be substituted 328 with one or more alternate amino acid residues to generate the one or more modified amino acid sequences 330. The generated amino acid (e.g., modified amino acid sequence) 330 can be provided to the predictive model 306 again for further classification. For instance, one or more features can be generated via feature processing 308 based on the characteristic data (not shown in FIG. 3) associated with the modified amino acid sequence 330. The classification 310 can classify, via the one or more processors, the modified amino acid sequence 330 as either a high-viscous amino acid sequence or a low-viscous amino acid sequence. The loop, from the predictive model 306 to determine the first set of high-viscous amino acid sequence (e.g., high-viscous amino acid sequence 316), to the generative model 318 to generate the modified amino acid sequence 330, and back to predictive model 306 to classify the modified amino acid sequence, can be repeated multiple times until the outputs of the predictive model comprise no high-viscous amino acid sequence.

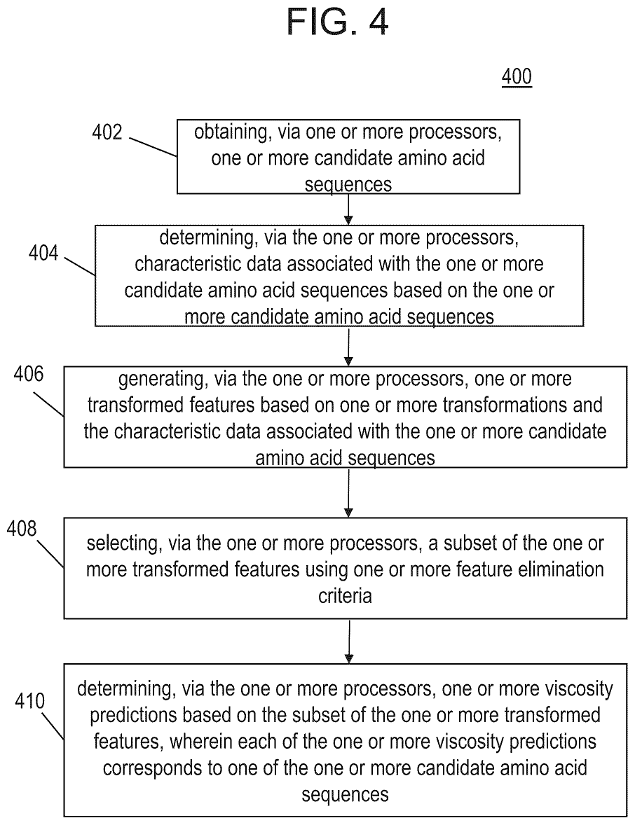

FIG. 4 is a flowchart of an illustrative method 400 for determining one or more viscosity predictions associated with one or more candidate amino acid sequences, in accordance with some embodiments of the technology described herein.

The method 400 can comprise a step 402 of obtaining, via one or more processors, one or more candidate amino acid sequences. The one or more candidate amino acid sequences can be obtained from one or more nucleic acid sequences. In some embodiments, the deoxyribonucleic acid (DNA) encodes one or more candidate amino acid sequences. For instance, the information in DNA can be transferred to a messenger ribonucleic acid (mRNA) via transcription, wherein the DNA of a gene can serve as a template for complementary base-pairing, and an enzyme called RNA polymerase II can catalyze the formation of a pre-mRNA molecule, which is then processed to form mature mRNA. Then the mRNA can be translated into one or more candidate amino acid sequences. In this situation, the details of the one or more candidate amino acid sequences can be determined from the sequence of DNA that encodes the one or more candidate amino acid sequences. In another example, the one or more candidate amino acid sequences can be produced by DNA cloning into plasmids and recombinant mammalian expression prior to purification.

The method 400 can comprise a step 404 of determining, via the one or more processors, characteristic data associated with the one or more candidate amino acid sequences based on the one or more candidate amino acid sequences. The characteristic data associated with the one or more candidate amino acid sequences can comprise at least one of a charge, an aromatic content, or a hydrophobicity score. The characteristic data associated with the one or more candidate amino acid sequences can comprise at least one of a charge, an aromatic content, or a hydrophobicity score in one or more amino acid sequence regions (e.g., Fv regions of mAbs, CDRs, frameworks). The characteristic data associated with the one or more candidate amino acid sequences can comprise an aromatic content. The one or more amino acid sequence regions can be selected based on the amino acid counts. For instance, in some embodiments, the one or more amino acid sequence regions comprise at least 1, 5, 10, 15, 20 or more amino acids. In some other embodiments, the one or more amino acid sequence regions comprise at most 20, 15, 10, 5 or less amino acids. The one or more amino acid sequence regions can be further filtered to select those that are likely to be informative based on their amino acid sequence patterns, for example, amino acid sequence patterns that impact the charge, aromatic content, and/or hydrophobicity score of the whole amino acid sequence. The characteristic data can be determined by computation via any mathematical/computational techniques based on the one or more candidate amino acid sequences described elsewhere herein. The characteristic data can be determined by any suitable analytical techniques described elsewhere herein.

The method 400 can comprise a step 406 of generating, via the one or more processors, one or more transformed features based on one or more transformations and the characteristic data associated with the one or more candidate amino acid sequences. The method may further comprise preprocessing the one or more features by applying one or more transformations including at least one of cleaning, centralizing, or scaling. The one or more transformations can include cleaning. The cleaning can comprise removing zero and near-zero variance features. The cleaning can comprise removing duplicated features and filtering the one or more features based on one or more filtering criteria. Details of the one or more filtering criteria are described elsewhere herein. The transformations can comprise binarization. For instance, feature values greater than 0 can be set to 1, such that feature values are either 0 or 1. In other embodiments, a smoothing function may be implemented (e.g., to provide more granular values) instead of binarization to 0 or 1.

The method 400 can comprise a step 408 of selecting, via the one or more processors, a subset of the one or more transformed features using one or more feature elimination criteria. The feature elimination criteria can comprise recursive feature elimination. The recursive feature elimination can comprise a feature selection method that fits a model and removes the weakest feature (or features) until the specified number of features is reached. The selecting a subset of the one or more transformed features can comprise any feature selection technique/algorithm. The feature selection technique/algorithm can comprise univariate selection, recursive feature elimination, principal component analysis, and feature importance.

The method 400 can comprise a step 410 of determining, via the one or more processors, one or more viscosity predictions based on the subset of the one or more transformed features, wherein each of the one or more viscosity predictions corresponds to one of the one or more candidate amino acid sequences. Prior to calculating the one or more viscosity predictions, the classifying can comprise generating one or more features based on one or more candidate amino acid sequences and the characteristic data associated with the one or more candidate amino acid sequences. In this situation, the characteristic data associated with the one or more candidate amino acid sequences can comprise at least one of amino acid counts, charge, aromatic content, or hydrophobicity score. The one or more candidate amino acid sequences and/or the characteristic data associated with the one or more candidate amino acid sequences can be input into the predictive model, and then one or more features can be generated based on the one or more candidate amino acid sequences and/or the characteristic data associated with the one or more candidate amino acid sequences. The one or more viscosity predictions can be determined based on the one or more features. Each of the one or more viscosity predictions corresponds to one of the one or more candidate amino acid sequences.

A modified amino acid sequence can be generated by the methods and systems disclosed herein. In some embodiments, the method can comprise obtaining, via one or more processors, a candidate amino acid sequence and characteristic data associated with the candidate amino acid sequence; classifying, via the one or more processors, the candidate amino acid sequence as a high-viscous amino acid sequence using a predictive model, wherein the predictive model calculates a viscosity prediction based on the candidate amino acid sequence and the obtained characteristic data and determines the candidate amino acid sequence as the high-viscous amino acid sequence based on the viscosity prediction and a predetermined viscosity threshold; and generating, via the one or more processors, the modified amino acid sequence based on the high-viscous amino acid sequence using a generative model, wherein the generative model identifies one or more non-interactive amino acid residues and substitutes the one or more non-interactive amino acid residues with one or more alternate amino acid residues to generate the modified amino acid sequence. The method can further comprise classifying the modified amino acid sequence using the predictive model. The candidate amino acid sequence can have the viscosity prediction higher than or equal to the predetermined viscosity threshold. The modified amino acid sequence can have a viscosity prediction lower than the predetermined viscosity threshold. The predetermined viscosity threshold can be 15 centipoise. The method can further comprise providing the modified amino acid sequence for laboratory experiments. Details of the characteristic data, the predictive model, the generative model, the predetermined viscosity threshold, the high-viscous amino acid sequence, the non-interactive amino acid residues, and the alternate amino acid residues are described elsewhere herein.

### EXAMPLES

Machine learning techniques were developed for generating one or more modified amino acid sequences. In some embodiments, the developed machine learning techniques utilized different features, generated based on one or more amino acid sequences, to determine one or more viscosity predictions of the one or more amino acid sequences.

MMAb3 is a fully human monoclonal antibody targeting huIL-13 that was discovered through immunization of a transgenic mouse and affinity matured using the huTARG mammalian display platform. The binding and functional profile of MMAb3 is 34 fM binding to huTL-13 and cross-reactivity to TL-13 from cynomolgus monkey. After MMAb3 was engineered to remediate hotspots (MMAb3.1), the predictive model correctly predicted that it had a significant probability of HIGH viscosity (e.g., classified as one of the first set of high-viscous amino acid sequences). MMAb3.1 was recombinantly produced from Chinese hamster ovary (CHO) cells and concentrated to 150 mg/mL in a platform buffer (10 mM acetate, 9% sucrose, pH 5.2), a viscosity of 33.8 cP was measured. To identify that the viscosity was not increased while engineering out hotspots, the viscosity of the parental MMAb3 molecule was measured, and it had a higher viscosity of 76.9 cP. Antibodies that bound to this epitope were rare, instead of culling the molecule, an engineering campaign to reduce viscosity while maintaining function was described.

### A. Predictive Model for Viscosity

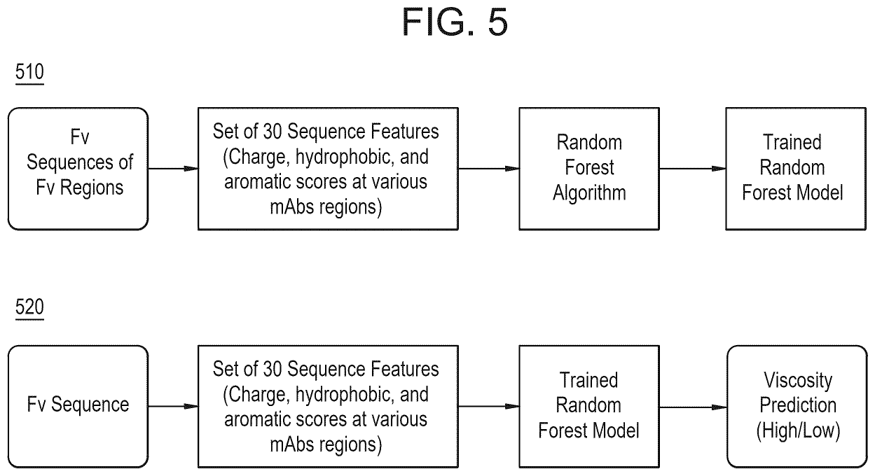

The predictive model was trained on a set of internally developed monoclonal antibodies (mAbs). The model was trained using a list of 206 hand-engineered features based on amino acid counts and calculated charge, aromatic content, and hydrophobicity in the various Fv regions of the mAbs (CDRs, framework, etc) as explained in flowchart 510 of FIG. 5. Preprocessing of features involved removing zero and near-zero variance features. A subset of 30 score-based features were selected in the predictive model. Model Selection and hyper-parameter tuning were applied to both linear and non-linear machine learning algorithms. The hyper-parameters were tuned using grid-search and 3-fold cross validation and optimized for accuracy. In order to assess model performance, the dataset was split into 70% train and 30% test set 10,000 times. For each split, hyper-parameter tuning was performed and model was evaluated on the test set using different metrics. The final model was selected based on the accuracy distribution and median value.

One example of predictive model was a Random Forest classifier (with number of randomly drawn candidate variables, or number of randomly drawn candidate variables, representing the number of features that are available to be considered at each split). The model development workflow was summarized in flowchart 520 of FIG. 5. To generate predictions for IL13 sequences, the predictive model first took the Fv sequence as input. It then computed 30 sequence-based descriptors which represent three types of scores: charge, aromatic, and hydrophobic on various Fv regions. The predictive model then passed these as input to a trained Random Forest model to get viscosity predictions as either LOW (e.g., one of the second set of low-viscous amino acid sequences) or HIGH viscosity (e.g., one of the first set of high-viscous amino acid sequences).

### B. Viscosity Predictions Determination and Modified Amino Acid Sequences Generation

To identify potential causes of viscosity, a Schrodinger BioLuminate Protein Preparation was performed on the PBD:7REW MMAb3 NHP IL-13 co-crystal structure, after the removal of the IL-13 antigen, with pH set to 5.2. This was followed by running Schrodinger Protein Surface Analysis. Resulting surface patches were filtered for significant impact based on a patch size of >500 Å² and a patch score of >500. In addition, residues were narrowed down to variants with a contribution score of >40 kcal/mol (see Table 1). The contribution score was calculated as an energy score based on the collection of elements on the protein surface. Amino acid interactions were analyzed and displayed using Pymol Schrodinger software.

TABLE 1
Schrodinger Surface Patch Analysis of Prominent Patches of MMAb3 from PDB:7REW
after removal of IL-13. Patches of smaller size or patch score were excluded.
For negatively charged patch 44, surface analysis revealed residues with the highest
contribution in kcal/mol were most prevalent in the light chain. Residues lacking
a negative charge generally had a significantly lower contribution score.

| Patch | Size | Patch | | | Contribution | Side Chain |
|---|---|---|---|---|---|---|
| ID | (Å2) | Score | Residue (ref) | Topo | (kcal/mol) | Accessibility |
| 44 | 614 | 776.47 | LC:E3 | LC:FR1 | 116.07 | 57.0% |
| | | | LC:D26 | LC:CDR1 | 100.87 | 58.2% |
| | | | LC:D33 | LC:CDR1 | 100.77 | 47.8% |
| | | | LC:D87 | LC:FR3 | 82.47 | 50.7% |
| | | | HC:E53 | HC:FR2 | 48.09 | 39.1% |
| | | | LC:Y2 | LC:FR1 | 46.86 | 51.7% |
| | | | LC:D67 | LC:CDR2 | 41.44 | 26.3% |
| | | | LC:S111 | LC:CDR3 | 28.28 | 63.4% |
| | | | LC:G32 | LC:CDR1 | 25.77 | 21.4% |
| | | | LC:K30 | LC:CDR1 | 22.34 | 41.2% |
| | | | LC:Y40 | LC:CDR1 | 21.72 | 40.0% |
| | | | LC:T5 | LC:FR1 | 20.59 | 48.2% |
| | | | LC:D110 | LC:CDR3 | 15.46 | 18.9% |
| | | | LC:N82 | LC:FR3 | 13.37 | 6.1% |
| | | | HC:E99 | HC:FR3 | 12.67 | 49.0% |
| | | | LC:T135 | LC:CDR3 | 12.58 | 34.0% |
| | | | LC:F139 | LC:FR4 | 10.98 | 7.4% |
| | | | LC:K39 | LC:CDR1 | 9.25 | 19.3% |
| | | | HC:L52 | HC:FR2 | 8.94 | 0.3% |
| | | | LC:S24 | LC:CDR1 | 8.35 | 46.2% |
| | | | LC:G84 | LC:FR3 | 7.6 | 108.3% |
| | | | LC:S1 | LC:FR1 | 6.01 | 34.8% |
| | | | LC:H58 | LC:CDR2 | 5.77 | 22.7% |
| | | | LC:V138 | LC:CDR3 | 5.16 | 20.0% |
| 9 | 512 | 542.25 | HC:K86 | HC:FR3 | 112.73 | 47.4% |
| | | | HC:R20 | HC:FR1 | 97.49 | 41.6% |
| | | | HC:R97 | HC:FR3 | 89.75 | 39.2% |
| | | | HC:N94 | HC:FR3 | 25.48 | 40.9% |
| | | | HC:R77 | HC:FR3 | 23.89 | 16.6% |
| | | | HC:S7 | HC:FR1 | 18.78 | 52.6% |
| | | | HC:T144 | HC:FR4 | 18.78 | 52.1% |
| | | | HC:G8 | HC:FR1 | 17.52 | 95.6% |
| | | | HC:S73 | HC:FR3 | 17.01 | 41.9% |
| | | | HC:S95 | HC:FR3 | 16.93 | 55.4% |
| | | | HC:Y90 | HC:FR3 | 16.56 | 19.2% |
| | | | HC:S149 | HC:FR4 | 16.33 | 56.3% |
| | | | HC:Q92 | HC:FR3 | 13.79 | 33.9% |
| | | | HC:T143 | HC:FR4 | 11.89 | 23.1% |
| | | | HC:S81 | HC:FR3 | 9.28 | 44.1% |
| | | | HC:D72 | HC:FR3 | 6.96 | 57.7% |
| | | | HC:P15 | HC:FR1 | 6.65 | 28.6% |
| | | | HC:G10 | HC:FR1 | 6.54 | 30.1% |
| | | | HC:S18 | HC:FR1 | 5.78 | 42.9% |
| | | | HC:S22 | HC:FR1 | 5.06 | 34.5% |
| | | | HC:A98 | HC:FR3 | 5.03 | 59.3% |

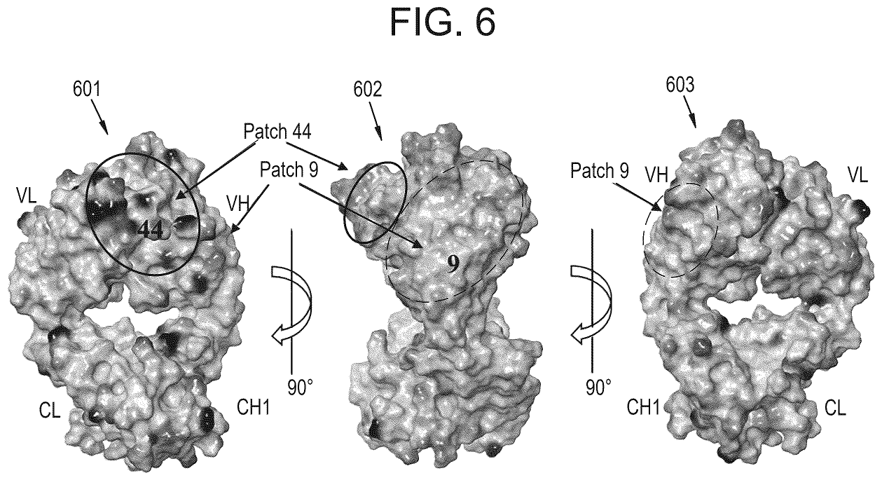

To identify the residues contributing to high viscosity, a co-crystal structure of the MMAb3 antigen-binding fragment (Fab) in complex with the NHP IL-13 target (PDB:7REW) at a resolution of 2.1 angstrom was generated. The structure was used to calculate protein surface features and target amino acid substitutions away from residues directly involved in the epitope-paratope interaction. To identify which residues to consider engineering, a protein surface analysis was done on PDB:7REW, without the IL-13 antigen, using the Schrodinger BioLuminate software package. Results of the surface analysis revealed an assortment of patches of various size and predicted impact. The patches with higher size and “patch scores” were visually inspected on the surface of the structure for compactness of residues with significant impact. From this inspection two patches were selected for potential engineering. Of the two patches, the largest one, “Patch 44” was a negatively charged patch positioned predominantly on the light chain, and the second largest patch, “Patch 9”, was positively charged and positioned predominantly on the heavy chain, see FIG. 6. FIG. 6 shows a surface view 601 of MMAb3 with residues contributing to negatively charged Patch 44 circled in solid line, a surface view 602 of MMAb3 after 90° rotation from 601 with residues contributing to Patch 44 circled in solid line and residues contributing to positively charged Patch 9 circled in dotted line, and a surface view 603 of MMAb3 after 90° rotation from 602 with contributing residues circled in dotted line. Negatively charged Patch 44 is in top center of the surface view 601 and upper left of the surface view 602. Positively charged Patch 9 is in the top center of the surface view 602 and upper left of the surface view 603. Amino acid residues that were key players to creating each surface patch, as measured by a calculated “contribution score” >40 kcal/mol were targeted for engineering (Table 1). Patch 44 comprises LC:Y2, LC:E3, LC:D26, LC:D33, LC:D67, LC:D87, and HC:E53. One residue in Patch 44 with a lower contribution score was also included (LC:D110), due to its potential to address an isomerization site in LC:CDR3.

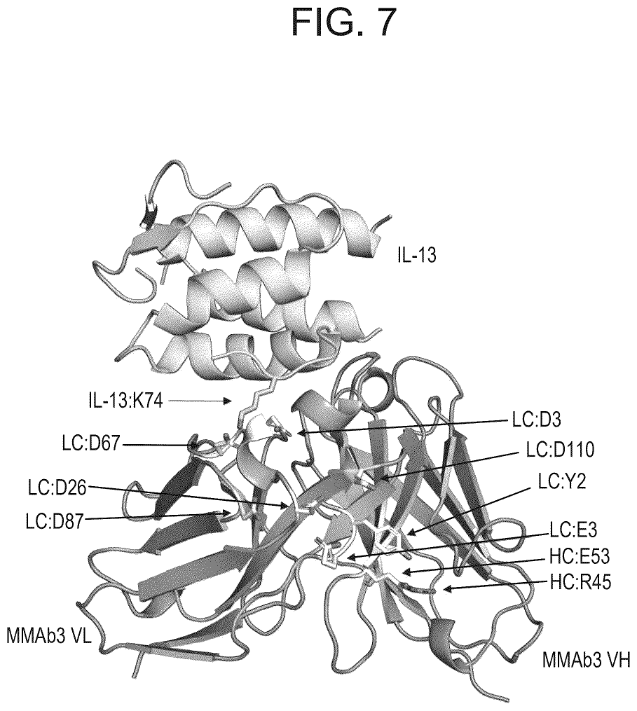

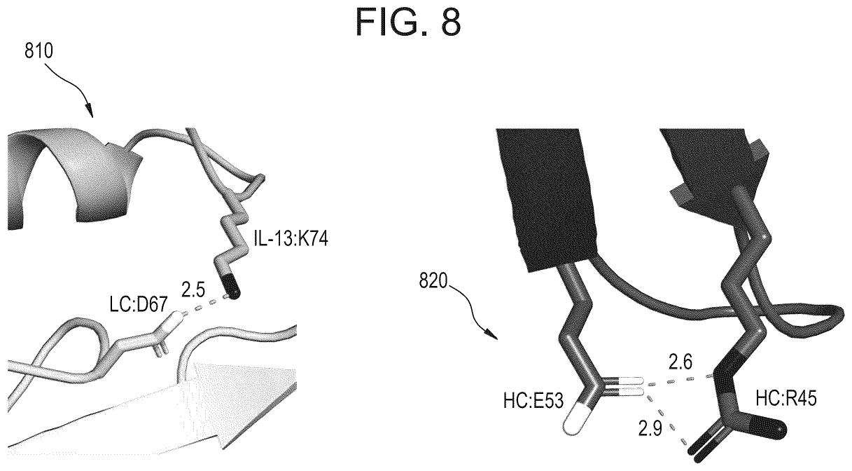

To focus protein engineering on non-critical residues, interactive residues were identified in the PDB:7REQ structure using the Protein Interaction Analysis function in BioLuminate software. Interactions in the structure were then visually confirmed using PyMOL software. FIG. 7 shows exemplary Pymol co-crystal of MMAb3 Fab with NHP IL-13 from PDB:7REW. In FIG. 7, IL-13 antigen is the upper molecule, MMAb3 LC is the lower left molecule, and MMAb3 HC is the lower right molecule. In FIG. 7, residues with significant contribution to negatively charged Patch 44 are in white color (shown on the right side of MMAb3 LC) demonstrating positioning of contributing residues across the LC of the Fab surface. Graph 810 of FIG. 8 demonstrates that MMAb3 LC:D67 interacts with IL-13 K74 with complementary charges and a proximity of 2.5 Å. Graph 820 of FIG. 8 demonstrates that MMAb3 HC:E53 interacts with the HC:R45, contributing to the beta sheet structure with a proximity of 2.6 and 2.9 Å. Of the residues in Patch 44 with high contribution scores, the HC:E53 residue was observed to contribute to the heavy chain (HC) structure by interacting with HC:R45 in the beta sheet (820 in FIG. 8) and as a result was not engineered. The LC:D67 residue was observed to interact directly with IL-13 residue K74, serving a critical role in IL-13 antigen binding (810 in FIG. 8). The interaction of LC:D67 with IL-13:K74 was also specified in the Schrodinger Protein Interaction Analysis (data not included). Due to the crucial interactive roles of these residues, they were not modified. To identify potential alternate residues with minimal immunogenicity risk, the MMAb3.1 variable domain was aligned to related light chain (LC) and heavy chain (HC) germlines, VL3 and VH3 respectively. For the LC, alignment revealed alternate sub-germline options of Y2S, E3V, D33S, D33K, D87N, and D87T. For the HC, most residues checked were highly conserved with the exception of HC:R97K.

A set of variants engineered for improved viscosity (e.g., one or more modified amino acid sequences) was narrowed down by entering their Fv sequences into the generative model. Potential mutations to lower viscosity were evaluated individually and in combinations of 2, 3, and 4 mutations (Table 2 and Table 3). For positively charged Patch 9 with contributing residues HC:R20, HC:K86, and HC:R97, results demonstrated that none of the individual or combinatorial mutations changed the prediction from HIGH to LOW viscosity (Table 2).

TABLE 2
In silico viscosity prediction of MMAb3.1 positive charge patch #9
engineering variants. Hotspots and WT residues listed in header.
Mutations and corresponding viscosity predictions listed within the table.

| HC:R20 | HC:K86 | HC:R97 | Predicted Viscosity |
|---|---|---|---|
| | | | HIGH |
| S | | | HIGH |
| E | | | HIGH |
| | S | | HIGH |
| | | S | HIGH |
| | | E | HIGH |
| S | | S | HIGH |
| E | | E | HIGH |
| S | S | | HIGH |
| | S | S | HIGH |
| S | S | S | HIGH |
| | E | | HIGH |
| E | E | | HIGH |
| | E | E | HIGH |
| E | E | E | HIGH |

This demonstrated a low probability of engineering success, and as a result corrections to the positively charged Patch 9 were not pursued any further. For negatively charged Patch 44, with contributing residues LC:Y2, LC:E3, LC:D26, LC:D33, LC:D87, and LC:D110, results demonstrated that single and double mutants as well as many mutations to polar or germnline residues predictions did not change from HIGH to LOW viscosity (Table 3). Combinations of three and four mutations from negatively charged residues to positively charged residues frequently did result in a change in predictions from HIGH to LOW viscosity, indicating a relatively high probability of engineering success (Table 3).

TABLE 3
In silico viscosity prediction of MMAb3.1 negative charge
patch # 44 engineered variants. Hotspots and WT
residues listed in header. Mutations and corresponding
viscosity predictions listed within the table. Variants
that were generated in lab for measurement are underlined.

| | | | | | | Predicted |
|---|---|---|---|---|---|---|
| LC:Y2 | LC:E3 | LC:D26 | LC:D33 | LC:D87 | LC:D110 | Viscosity |
| | | | | | | HIGH |
| S | | | | | | HIGH |
| | V | | | | | HIGH |
| | | K | | | | HIGH |
| | | | K | | | HIGH |
| | | | | N | | HIGH |
| | | | | | K | HIGH |
| S | | K | | | | HIGH |
| S | | | K | | | HIGH |
| S | | | | N | | HIGH |
| S | | | | K | | HIGH |
| S | | | | | K | HIGH |
| | V | K | | | | HIGH |
| | V | N | | | | HIGH |
| | V | S | | | | HIGH |
| | V | | K | | | HIGH |
| | V | | S | | | HIGH |
| | V | | | N | | HIGH |
| | V | | | K | | HIGH |
| | V | | | | K | HIGH |
| | | K | K | | | HIGH |
| | | N | K | | | HIGH |
| | | K | S | | | HIGH |
| | | K | | N | | HIGH |
| | | K | | K | | HIGH |
| | | N | | N | | HIGH |
| | | K | | T | | HIGH |
| | | K | | | K | HIGH |
| | | | K | N | | HIGH |
| | | | K | K | | HIGH |
| | | | S | N | | HIGH |
| | | | K | T | | HIGH |
| | | | K | | K | HIGH |
| | | | | N | K | HIGH |
| | | | | K | K | HIGH |
| S | | K | K | | | HIGH |
| S | | K | | N | | HIGH |
| S | | K | | | K | HIGH |
| S | | | K | N | | HIGH |
| S | | | K | | K | HIGH |
| S | | | | N | K | HIGH |
| | V | K | K | | | LOW |
| | V | N | K | | | HIGH |
| | V | S | K | | | HIGH |
| | V | K | S | | | HIGH |
| | V | N | S | | | HIGH |
| | V | K | | N | | HIGH |
| | V | K | | K | | HIGH |
| | V | K | | | K | LOW |
| | V | | K | N | | HIGH |
| | V | | K | K | | HIGH |
| | V | | K | | K | LOW |
| | V | | | N | K | HIGH |
| | V | | | K | K | HIGH |
| | | K | K | N | | LOW |
| | | N | K | N | | HIGH |
| | | K | S | N | | HIGH |
| | | K | K | T | | LOW |
| | | N | S | T | | HIGH |
| | | K | K | | K | LOW |
| | | K | | N | K | LOW |
| | | K | | K | K | LOW |
| | | | K | N | K | LOW |
| | | | K | K | K | LOW |
| | V | | K | N | K | LOW |
| | V | | K | K | K | LOW |
| | V | K | | N | K | LOW |
| | V | K | | K | K | LOW |
| | V | K | K | | K | LOW |
| | V | K | K | N | | LOW |
| | V | K | K | K | | LOW |
| | | K | K | K | K | LOW |
| | | K | K | N | K | LOW |
| | | K | K | K | K | LOW |

In many combinatorial cases, mutations at LC:E3 and LC:D87 had the same prediction, whether substituted with the positively charged amino acid Lysine or alternate sub-germline residues LC:E3V and LC:D87N, respectively. At these sites, germline residues were advanced to minimize potential immunogenicity. Altogether, the probability of 74 candidate mutation combinations were assessed with the predictive model. From the viscosity predictions of mutations (e.g., modified amino acid sequences), 20 variants were identified to predict to have LOW viscosity. Of those 20 variants, by limiting mutations at LC:E3 and LC:D87 to germline residues, variants were further narrowed in consideration down to 11. Six wild type or single mutation variants and 3 triple mutants were added, each predicted to have HIGH viscosity were also included for production followed by cone and plate viscosity measurement.

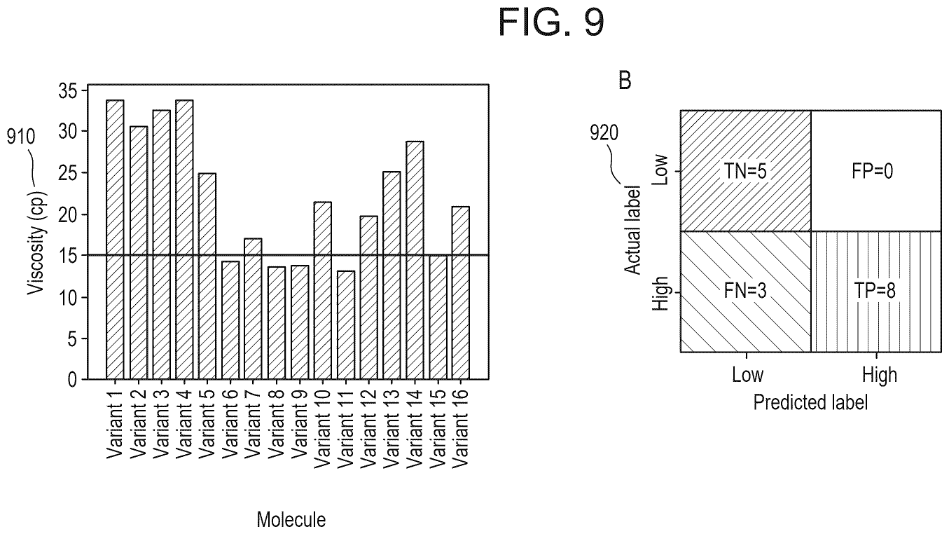

Of the 20 variants advanced into production, 4 variants failed to express, leaving 16 variants for analysis. Cone and plate viscosity measurements were taken of the purified MMAb3.1 variants concentrated at 150 mg/mL: 5 variants had viscosity measured at <15 cP. The distribution of measured viscosity for these 16 MMAb3.1 variants is shown in graph 910 of FIG. 9. Furthermore, per variant measured viscosity versus prediction is shown in detail in Table 4. Table 5 summarizes the different performance metrics (the maximum value for accuracy is 100% and for all other metrics is 1; a larger value of these metrics indicates better performance). The accuracy of model prediction for the 16 tested variants was 81.25%. The predictive model achieved a precision of 1: all the engineered variants that were predicted to have HIGH viscosity had cone and plate measurements in the range of 19.8 to 76.9 cP at 150 mg/mL (FIG. 9 and Table 4). In graph 920 of FIG. 9, confusion matrix summarizing the performance on the IL13 dataset (TNs True Negative (model predicted low and it's true), FP=False Positive (model predicted high and it's false i.e., actual measurement value is low), FN=False Negative (model predicted low and it's false i.e., actual measurement value is high), TP=True Positive (model predicted high and it's true)). 11 of the 16 variants had measured viscosity of ≥15 cP at 150 mg/mL. Of these, the model incorrectly predicted 3 to be LOW viscosity, hence achieving a recall of 0.73. The overall tradeoff between precision and recall was represented with the F-1 score of 0.84 (equal to the harmonic mean of precision and recall, Table 5).

TABLE 4
Results of viscosity prediction, viscosity measure in cP, and functional activity in pM for
lead variants. Concentration at which viscosity measures were taken are listed in mg/mL.

| | | | | | Visc. | Conc. | Visc. | TARC IC50 |
|---|---|---|---|---|---|---|---|---|
| LC:E3 | LC:D26 | LC:D33 | LC:D87 | LC:D110 | Prediction | (mg/mL) | (cP) | Ave. (pM) |
| | | | | | HIGH | 150 | 33.8 | 39.28 |
| V | | | | | HIGH | 150 | 30.7 | |
| | | K | | | HIGH | 157 | 32.6 | |
| | | | N | | HIGH | 150 | 34 | |
| | | | | K | HIGH | 145 | 25 | |
| | | K | N | K | LOW | 150 | 14.3 | 51.36 |
| | K | | N | K | LOW | 150 | 13.1 | |
| | K | K | | K | LOW | 150 | 13.7 | |
| | K | K | N | | LOW | 151 | 13.8 | 28.54 |
| V | | | N | K | HIGH | 150 | 21.6 | |
| V | | K | | K | LOW | 145 | 13.1 | 52.16 |
| V | | K | N | | HIGH | 154 | 19.8 | |
| V | K | | | K | LOW | 150 | 25.2 | |
| V | K | | N | | HIGH | 153 | 28.8 | |
| V | | K | N | K | LOW | 150 | 14.9 | |
| V | K | | N | K | LOW | 155 | 20.9 | |

TABLE 5
Different metrics quantifying the model performance.
Maximum value for accuracy is 100% and for all
other metrics it is 1. Higher value of these metrics
denotes better performance.

| Metrics | Formulae | Value |
|---|---|---|
| Accuracy | (TP + TN)/(TP + TN + FP + FN) | 81.25% |
| Precision | TP/(TP + FP) | 1 |
| Recall | TP/(TP + FN) | 0.73 |
| F1-score | 2 * (Precision * Recall)/ | 0.84 |
| | (Precision + Recall) | |

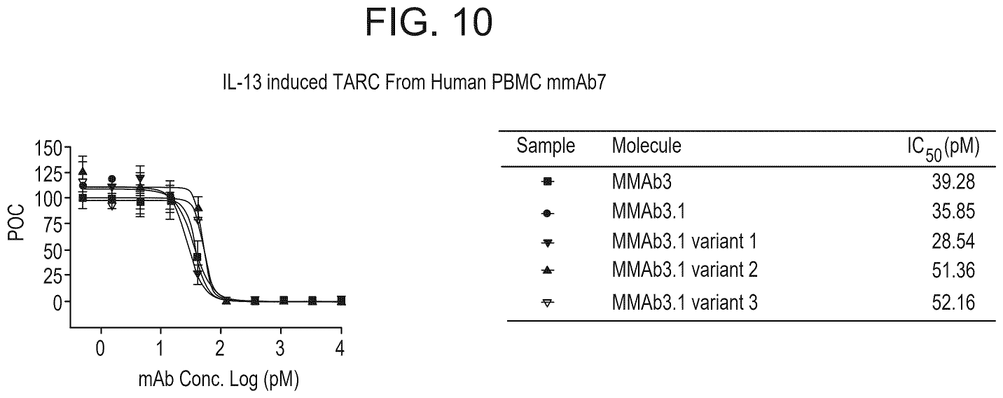

Three engineered variants with low viscosity were tested for retained function in a human PBMC, IL-13 induced TARC assay, in which a serial dilution of candidate molecules have a dose response inhibition IL-13 induced TARC from PBMCs. Activity of the low viscosity mutants, measured as IC50 of TARC MSD signal was within 2-fold of the MMAb3 and MMAb3.1 controls of 35.85 and 39.28 pM respectively and ranged from 28.54 to 52.16 pM (FIG. 10, Table 4). FIG. 10 shows functional assessment of lead candidates in the human PBMC, IL-13-induced TARC assay. Human IL-13 was pre-incubated with serially diluted mAb samples, then added to PBMCs and incubated for 48 h. TARC (CCL17) detection was measured from cell supernatants using the MSD platform with results normalized and graphed as percent of control (POC) for the inhibition of IL-13 induced TARC (CCL17). Results demonstrate an IC50 for viscosity engineered variants that is within range of the MMAb3 and MMAb3.1 parental clones.

### Materials and Lab Procedures

Candidate molecules (e.g., candidate amino acid sequences) were produced by DNA cloning into plasmids and recombinant mammalian expression prior to purification by Protein A capture. DNA plasmids were cloned using golden gate technology to assemble antibody variable domains, purchased as G-block nucleotide strands, with IgG constant domains and insert them into proprietary recombinant expression vectors. In the HC containing plasmid, the selectable marker was the puromycin resistance gene, puromycin-N-acetyltransferase. In the LC containing plasmid, the selectable marker was the hygromycin resistance gene, hygromycin B phosphotransferase. Plasmids were transfected into a CHO—K1 cell line for expression using Lipofectamine LTX (Thermo Fisher Scientific) by established lipid transfection methods with equal parts of mAb encoding plasmid to Piggybac transposase encoding plasmid (System Biosciences). After recovery from selection by puromycin at 20 μg/mL and hygromycin at 500 μg/mL (Thermo Fisher Scientific), cultures were scaled by dilution into a proprietary growth medium. Inoculation was performed by dilution into a proprietary front-loaded production medium at a density of 1.5×10⁶ cells/mL before production for 6 days. Clarified supernatants were affinity captured by MabSelect SuRe chromatography (Cytia, Piscataway, NJ), using Dulbecco's PBS without divalent cations (Invitrogen, Carlsbad, CA) and 25 mM tris-HCl, 500 mM L-Arg-HCl, pH 7.5 as the wash buffers and 100 mM acetic acid, pH 3.6 as the elution buffer. All separations were carried out at ambient temperature. When the absorbance at 280 nm was above a defined threshold, the elution was immediately conditioned with 0.03-volumes of 2 M tris using an in-line mixer. Conditioning was terminated when the absorbance was below the defined threshold. The affinity pool was filtered through a 0.22 μm cellulose acetate filter.

The pools were diafiltered against approximately 30 volumes of 10 mM sodium acetate, 9% sucrose, pH 5.2 using Slide-A-Lyzer dialysis cassettes with a 20 kDa cutoff membrane (Thermo Scientific, Waltham, MA) and further concentrated using Centriprep centrifugal concentrator with a 30 kDa cutoff membrane (EMD Millipore, Burlington, MA). The concentrated material was then filtered through a 0.8/0.2 m cellulose acetate filter and the concentration was determined by the absorbance at 280 nm using the calculated extinction coefficient. Sample purity was determined by LabChip GXII analysis under reducing (with 32.7 mM) and non-reducing (with 25 mM iodoacetamide) conditions (FIG. 9). Analytical SEC was carried out using a BEH200 column (Waters, Milford, MA) with an isocratic elution in 100 mM sodium phosphate, 50 mM NaCl, 7.5% ethanol, pH 6.9 over 10′.

Prior to cone and plate viscosity measurements, Polysorbate 80 was added to a final concentration of 0.01%. Cone and plate viscosity measurements were performed at 25C on an Anton Paar Rheometer using standard methods.

Variants of MMAb3.1 were tested for retained function using a human PBMC IL-13 induced TARC assay. Peripheral blood mononuclear cells (PBMC) were isolated from leukapheresis packs (HemaCare Corp) by density centrifugation with Ficoll-Pague Plus™ kits (Millipore Sigma Aldrich). PBMC were resuspended in CPZ™ cryopreservation medium (Incell) at a cell density of 2×10⁸ cells/mL before being frozen down. To test inhibition of IL-13 induced CCL17/TARC by MMAb3.1 variants, PMBCs were thawed and resuspended in assay media containing RPMI-1640, 10% heat inactivated FBS, and Penicillin-Streptomycin-Glutamine. 2×10⁵ cells/well were added to 96 well U-bottom tissue culture plates (Costar) then incubated for 2 hours at 37° C. and 5% CO₂ to recover. MMAb3.1 variants underwent a ten-point 1:3 serial dilution (100 nM to 0.005 nM) and equal volume were pre-incubated with 30 ng/mL Human IL-13 (Pepro Tech) for 20 minutes at room temperature. Resulting pre-incubates were added at a 1:5 ratio to 2×10⁵ PBMC and incubated for an additional 48 hours at 37° C., 5% CO₂. 50 μL supernatant was collected and added to CCL17/TARC plates (V-PLEX Plus, Meso Scale Diagnostics) and CCL17/TARC was detected following the manufacturer's instructions on a MESO SECTOR S 600 MSD plate reader (Meso Scale Diagnostics). IC₅₀ values were attained with GraphPad Prizm software by determining averages and standard deviations of duplicate samples and performing a non-linear curve fit analysis with a four-parameter variable slope.

An illustrative implementation of a computer system 1100 that may be used in connection with any of the embodiments of the technology described herein (e.g., such as the methods of FIGS. 2 and 4) is shown in FIG. 11. The computer system 1100 includes one or more processors 1110 and one or more articles of manufacture that comprise non-transitory computer-readable storage media (e.g., memory 1120 and one or more non-volatile storage media 1130). The processor 1110 may control writing data to and reading data from the memory 1120 and the non-volatile storage device 1130 in any suitable manner, as the aspects of the technology described herein are not limited to any particular techniques for writing or reading data. To perform any of the functionality described herein, the processor 1110 may execute one or more processor-executable instructions stored in one or more non-transitory computer-readable storage media (e.g., the memory 1120), which may serve as non-transitory computer-readable storage media storing processor-executable instructions for execution by the processor 1110.

Computer device 1100 may also include a network input/output (I/O) interface 1140 via which the computing device may communicate with other computing devices (e.g., over a network), and may also include one or more user I/O interfaces 1150, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

1. A computer-implemented method for generating one or more modified amino acid sequences, comprising:
(a) obtaining, via one or more processors, one or more candidate amino acid sequences and characteristic data associated with the one or more candidate amino acid sequences;
(b) classifying, via the one or more processors, the one or more candidate amino acid sequences into a first set of high-viscous amino acid sequences and a second set of low-viscous amino acid sequences using a predictive model, the classifying comprising:
i. calculating one or more viscosity predictions based on the one or more candidate amino acid sequences and the obtained characteristic data; and
ii. determining the first set of high-viscous amino acid sequences and the second set of low-viscous amino acid sequences based on the one or more viscosity predictions and a predetermined viscosity threshold, wherein the first set of high-viscous amino acid sequences comprises at least one candidate amino acid sequence; and
(c) generating, via the one or more processors, the one or more modified amino acid sequences based on the first set of high-viscous amino acid sequences using a generative model, wherein at least one of the one or more modified amino acid sequences has a viscosity prediction lower than the predetermined viscosity threshold.

2. The computer-implemented method of claim 1, wherein the characteristic data associated with the one or more candidate amino acid sequences comprises at least one of a charge, an aromatic content, or a hydrophobicity score.

3. The computer-implemented method of claim 1, wherein the classifying further comprises, prior to calculating the one or more viscosity predictions, generating one or more features based on the characteristic data associated with the one or more candidate amino acid sequences.

4. The computer-implemented method of claim 3, wherein the classifying further comprises preprocessing the one or more features by applying one or more transformations including at least one of cleaning, centralizing, or scaling.

5. The computer-implemented method of claim 4, wherein the one or more transformations include cleaning, and wherein the cleaning comprises removing zero and near-zero variance features.

6. The computer-implemented method of claim 4, wherein the classifying further comprises selecting a subset of the one or more transformed features via recursive feature elimination.

7. The computer-implemented method of claim 1, wherein the predictive model comprises a random forest model.

8. The computer-implemented method of claim 1, wherein the predetermined viscosity threshold is 15 centipoise.

9. The computer-implemented method of claim 1, wherein each of the one or more viscosity predictions corresponds to one of the one or more candidate amino acid sequences.

10. The computer-implemented method of claim 1, wherein at least one of the first set of high-viscous amino acid sequences has a viscosity prediction higher than or equal to the predetermined viscosity threshold.

11. The computer-implemented method of claim 1, wherein the second set of low-viscous amino acid sequences comprises at least one candidate amino acid sequence, and wherein at least one of the second set of low-viscous amino acid sequences has a viscosity prediction lower than the predetermined viscosity threshold.

12. The computer-implemented method of claim 1, wherein the generating the one or more modified amino acid sequences using the generative model comprises:
generating one or more amino acid structures based on the first set of high-viscous amino acid sequences;
identifying one or more non-interactive amino acid residues based on the one or more amino acid structures; and
substituting the one or more non-interactive amino acid residues with one or more alternate amino acid residues to generate the one or more modified amino acid sequences.

13. The computer-implemented method of claim 1, further comprising providing the second set of low-viscous amino acid sequences for laboratory experiments.

14. The computer-implemented method of claim 1, further comprising classifying the one or more modified amino acid sequences using the predictive model.

15-19. (canceled)

20. A computer system for generating one or more modified amino acid sequences, comprising:
a memory storing instructions; and
one or more processors configured to execute the instructions to perform operations including:
(a) obtaining one or more candidate amino acid sequences and characteristic data associated with the one or more candidate amino acid sequences;
(b) classifying the one or more candidate amino acid sequences into a first set of high-viscous amino acid sequences and a second set of low-viscous amino acid sequences using a predictive model, the classifying comprising:
i. calculating one or more viscosity predictions based on the one or more candidate amino acid sequences and the obtained characteristic data; and
ii. determining the first set of high-viscous amino acid sequences and the second set of low-viscous amino acid sequences based on the one or more viscosity predictions and a predetermined viscosity threshold, wherein the first set of high-viscous amino acid sequences comprises at least one candidate amino acid sequence; and
(c) generating the one or more modified amino acid sequences based on the first set of high-viscous amino acid sequences using a generative model, wherein at least one of the one or more modified amino acid sequences has a viscosity prediction lower than the predetermined viscosity threshold.

21-30. (canceled)

31. The computer system of claim 20, wherein the generating the one or more modified amino acid sequences using the generative model comprises:
generating one or more amino acid structures based on the first set of high-viscous amino acid sequences;
identifying one or more non-interactive amino acid residues based on the one or more amino acid structures; and
substituting the one or more non-interactive amino acid residues with one or more alternate amino acid residues to generate the one or more modified amino acid sequences.

32. The computer system of claim 20, further comprising providing the second set of low-viscous candidate amino acid sequences for laboratory experiments.

33. The computer system of claim 20, further comprising classifying the one or more modified amino acid sequences using the predictive model.

34. A non-transitory computer readable medium for use on a computer system containing computer-executable programming instructions for performing a method of generating one or more modified amino acid sequences, the method comprising:
(a) obtaining one or more candidate amino acid sequences and characteristic data associated with the one or more candidate amino acid sequences;
(b) classifying the one or more candidate amino acid sequences into a first set of high-viscous amino acid sequences and a second set of low-viscous amino acid sequences using a predictive model, the classifying comprising:
i. calculating one or more viscosity predictions based on the one or more candidate amino acid sequences and the obtained characteristic data; and
ii. determining the first set of high-viscous amino acid sequences and the second set of low-viscous amino acid sequences based on the one or more viscosity predictions and a predetermined viscosity threshold, wherein the first set of high-viscous amino acid sequences comprises at least one candidate amino acid sequence; and
(c) generating the one or more modified amino acid sequences based on the first set of high-viscous amino acid sequences using a generative model, wherein at least one of the one or more modified amino acid sequences has a viscosity prediction lower than the predetermined viscosity threshold.

35-46. (canceled)

47. The non-transitory computer readable medium of claim 34, further comprising classifying the one or more modified amino acid sequences using the predictive model.

48-53. (canceled)
